(19) United States
(12) Patent Application Publication
(10) Pub. No.: US 2024/0352499 A1
(43) Pub. Date: Oct. 24, 2024

(54) POLYNUCLEOTIDES ENCODING NOVEL NUCLEASES, COMPOSITIONS THEREOF AND METHODS THEREOF FOR ELIMINATING DNA FROM PROTEIN PREPARATIONS

(71) Applicant: DANISCO US INC., Palo Alto, CA (US)

(72) Inventors: Cristina Bongiorni, Fremont, CA (US); Robert Pratt, II, San Jose, CA (US); Matt Reboli, Palo Alto, CA (US); Anita Van Kimmenade, Woodside, CA (US); Amanda Iverson, Mountain View, CA (US)

(21) Appl. No.: 18/683,749

(22) PCT Filed: Aug. 19, 2022
(86) PCT No.: PCT/US2022/075210

§ 371 Date: Feb. 14, 2024

Related U.S. Application Data
(60) Provisional application No. 63/235,345, filed on Aug. 20, 2021.

(51) Int. Cl. C12P 21/02; C12N 1/08; C12N 9/22; C12N 15/75; C12R 1/125

(52) U.S. Cl. CPC C12P 21/02; C12N 1/08; C12N 9/22; C12N 15/75; C12R 2001/125

(57) ABSTRACT

The instant disclosure provides, among other things, novel nuclease proteins (enzymes), novel nuclease compositions and protein preparations thereof, recombinant polynucleotides (DNA) encoding such nuclease proteins, recombinant host cells expressing and producing one or more nuclease proteins (optionally co-expressing one or more proteins of interest) and the like. More particularly, the nuclease proteins (enzymes) of the instant disclosure are particularly useful in mitigating DNA contamination, such as contaminating DNA present in a fermentation broth in which microbial host cells have been fermented, contaminating DNA present in recovered proteins of interest, contaminating DNA present in protein preparations, and the like.

### FIELD

The present disclosure is generally related to the fields of microbial cells, molecular biology, fermentation, protein production, protein recovery, protein preparations and the like. Certain embodiments of the disclosure are related to polynucleotides encoding novel proteins having nuclease activity, recombinant microbial cells expressing one or more heterologous nuclease proteins, microbial host cells producing proteins of interest, protein preparations derived therefrom essentially free from contaminating DNA, and the like.

### REFERENCE TO A SEQUENCE LISTING

The contents of the electronic submission of the text file Sequence Listing, named “NB41859-US-PSP_SequenceListing.txt” was created on Aug. 18, 2021 and is 36 KB in size, which is hereby incorporated by reference in its entirety.

### BACKGROUND

Microbial cells (strains) are particularly useful protein production hosts, as they can be easily grown at a large scale in relatively simple media. Thus, microbial (host) cells (e.g., bacterial cells, yeast cells, filamentous fugal cells and the like) are often used for recombinant production of industrial relevant proteins (e.g., amylases, proteases, cellulases, phytases, lactases, etc.) and protein biologics (e.g., antibodies, cytokines, receptors, etc.) used in animal feed, food enzymes, laundry, textile processing, grain processing, medical instrument cleaning, biotechnology industries, pharmaceutical industries and the like.

As appreciated by one of skill in the art, the large scale fermentation of microbial cells for the production of proteins can result in the release of contaminating DNA (e.g., genomic DNA, recombinant DNA) into the fermentation broth, which DNA polymers can cause broth viscosity problems, can interfere with subsequent protein recovery steps, can contaminate the final protein product(s), etc. Likewise, there are increasing regulatory restrictions with regard to the amount of recombinant DNA (rDNA) permitted in protein/enzyme preparations intended for use in animal feeds and/or use as human food additives. Most recently, the European Food Safety Authority (EFSA) has proposed limits of 10 ng rDNA (EFSA, 2018).

For example, PCT Publication No. WO1999/050389 generally describes microbial strains having DNA constructs encoding modified nuclease enzymes which are secreted into the periplasm or growth medium in an amount effective to enhance recovery of polymers, particularly for use in high cell density fermentation processes. PCT Publication No. WO2008/065200 describes the construction of a Bacillus strain expressing a Bacillus sp. nuclease gene (nucB) under the control of a phosphate regulated promoter (pstS). As generally described in this publication, the recombinant strain which expressed the nucB gene by the pstS promoter could be activated at the end of fermentation and express the nuclease (NucB) when it is needed for cleaning the fermentation broth for excess DNA. PCT Publication No. WO2011/010094 describes a method of removing nucleic acid contamination in reverse transcription and amplification reactions. US Patent Publication No. US2012/013549 describes a method for producing a nuclease from a Gram-negative bacterium (Serratia marcescens) by expressing the nuclease in a Gram-positive bacterium and secreting the nuclease into the medium. PCT Publication No. WO2013/043860 describes certain methods for reducing the DNA content of fermentation broths in which filamentous fungal cells have been cultivated. PCT Publication No. WO2019/081721, describes variant nucleases considered particularly useful in certain detergent formulations and the like.

Based on the foregoing, there remain ongoing and unmet needs in the art for nuclease proteins (enzymes), compositions thereof, uses thereof and/or methods thereof. As described hereinafter, the instant disclosure provides, among other things, novel nuclease proteins (enzymes), nuclease compositions and protein preparations thereof, recombinant polynucleotides (DNA) encoding such nuclease proteins, recombinant host cells expressing and producing one or more nuclease proteins (optionally co-expressing one or more proteins of interest) and the like. More particularly, as set forth and described below, the nuclease proteins (enzymes) of the instant disclosure are particularly useful in mitigating DNA contamination, e.g., such as contaminating DNA present in a fermentation broth in which microbial host cells have been fermented, contaminating DNA present in recovered proteins of interest, contaminating DNA present in protein preparations, and the like.

### SUMMARY

As generally known in the art, the presence of contaminating DNA (e.g., genomic DNA, recombinant DNA) in microbial cell fermentation broths and/or its presence in any down-stream protein recovery processes thereof, can lead to undesirable protein product qualities. As described hereinafter, certain embodiments of the disclosure are related to, among other things, the identification, isolation and characterization of novel genes encoding novel proteins having nuclease activity, recombinant microbial cells producing various protein products (e.g., proteins of interest) essentially free from DNA, compositions and methods for constructing such recombinant (genetically modified) microbial host cells, compositions and methods of producing and recovering various protein products essentially free from DNA, compositions and methods for rendering protein preparations essentially free from DNA and the like. More particularly, the novel nuclease proteins described herein are particularly suitable for degrading contaminating DNA which may be present in microbial fermentation broths, protein preparations, isolated proteins of interest and the like, such as protein biologics, animal feed proteins, human food enzymes, etc.

Thus, certain embodiments of the disclosure are directed to isolated nucleic acids (polynucleotides) comprising at least 80% identity to a nucleotide sequence of SEQ ID NO: 1, SEQ ID NO: 3, SEQ ID NO: 5, SEQ ID NO: 7, SEQ ID NO: 9, SEQ ID NO: 11 or SEQ ID NO: 15. In other embodiments, an isolated nucleic acid of the disclosure comprises at least 80% identity to a nucleotide sequence of SEQ ID NO: 1, SEQ ID NO: 3, SEQ ID NO: 5, SEQ ID NO: 7, SEQ ID NO: 9 or SEQ ID NO: 11, and encodes a protein comprising at least 85% identity to a protein of SEQ ID NO: 2, SEQ ID NO: 4, SEQ ID NO: 6, SEQ ID NO: 8, SEQ ID NO: 10 or SEQ ID NO: 12, respectively. In related embodiments, the encoded protein comprises deoxyribonuclease (DNase) activity. In other embodiments, the encoding protein comprising DNase activity is substantially protease resistant.

Thus, other embodiments of the disclosure are related to plasmids, vectors or expression cassettes comprising a polynucleotide sequence encoding protein comprising at least 85% identity to a protein of SEQ ID NO: 2, SEQ ID NO: 4, SEQ ID NO: 6, SEQ ID NO: 8, SEQ ID NO: 10 SEQ ID NO: 12, SEQ ID NO:16 or SEQ ID NO: 17.

In other embodiments, the disclosure provides isolated proteins comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17. In particular embodiments, an isolated protein comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17, comprises deoxyribonuclease (DNase) activity. In other embodiments, an isolated protein comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17, comprises a HNH nuclease superfamily domain. In other embodiments, an isolated protein comprises DNase activity and is substantially protease resistant. Certain other embodiments of the disclosure are therefore related to protein preparations comprising one or more proteins comprising DNase activity.

In another embodiment, the disclosure is related to a polynucleotide (e.g., an expression cassette) comprising an upstream (5′) promoter sequence operably linked to a downstream (3′) nucleic acid sequence encoding a protein comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17. In related embodiments, the polynucleotide comprises a terminator sequence positioned downstream (3′) and operably linked to the nucleic acid sequence encoding the protein comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17. In certain other embodiments, the disclosure is related to a polynucleotide comprising an upstream (5′) promoter sequence operably linked to a downstream (3′) nucleic acid sequence encoding protein signal sequence operably linked to a downstream (3′) nucleic acid sequence encoding a protein comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17. In another embodiment, the polynucleotide comprises a terminator sequence positioned downstream (3′) and operably linked to the nucleic acid sequence encoding the protein comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17.

Certain other embodiments of the disclosure are directed to recombinant microbial cells (strains) expressing one or more proteins comprising at least 85% identity to a protein sequence of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17. In certain embodiments, the microbial cell is selected from the group consisting of a Gram-negative bacterial cell, a Gram-positive bacterial cell, a filamentous fungal cell or a yeast cell. In other embodiments, a recombinant microbial cell co-expresses (i) a protein of interest (POI) and (ii) one or more proteins comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17. In certain embodiments, the microbial cell is selected from the group consisting of a Gram-negative bacterial cell, a Gram-positive bacterial cell, a filamentous fungal cell or a yeast cell.

In related embodiments, a protein of interest (POI) is selected from the group consisting of a lyase, a ligase, a hydrolase, an oxidoreductase, a transferase, an isomerase, an antibody, a receptor, and a cytokine.

In other embodiments, the POI is an enzyme selected from the group consisting of acetyl esterases, aminopeptidases, amylases, arabinases, arabinofuranosidases, arylesterases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, lysozymes, mannosidases, nucleases, oxidases, oxidoreductases, pectate lyases, pectin acetyl esterases, pectin depolymerases, pectin methyl esterases, pectinolytic enzymes, perhydrolases, phytases, polyol oxidases, peroxidases, phenoloxidases, phytases, polyesterases, polygalacturonases, proteases, peptidases, rhamno-galacturonases, ribonucleases, transferases, transport proteins, transglutaminases, xylanases and hexose oxidases. In other embodiments, the POI is an animal feed protein or a food enzyme.

In yet another embodiment, the disclosure is directed to a fermentation broth obtained by fermenting a microbial cell expressing one or more proteins comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17. In certain other embodiments, the disclosure is directed to a fermentation broth obtained by fermenting a microbial cell co-expressing (i) one or more proteins of interest and (ii) one or more proteins comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17. In other embodiments, the broth is subjected to at least one the protein recovery step. In certain embodiments, the at least one the protein recovery step is selected from the group consisting of a cell lysis step, a cell separation step, a protein concentration step and a protein purification step.

Thus, certain other embodiments of the disclosure are related to, among other things, methods for producing various protein products (e.g., proteins of interest) essentially free from DNA, methods for recovering various protein products essentially free from DNA, methods for rendering protein preparations essentially free from DNA, methods for constructing recombinant (genetically modified) microbial host cells heterologously expressing one or more proteins comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17, and the like.

Certain embodiments are therefore related to methods for producing a protein of interest (POI) essentially free from contaminating DNA comprising (a) obtaining or constructing a microbial cell expressing a POI and modifying the cell to express one or more proteins comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17, and (b) fermenting the modified cell under suitable conditions for the expression of the POI and the one or more proteins comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17, wherein the POI produced is essentially free from contaminating DNA. In certain embodiments, the expressed POI is retained inside (intracellular) the microbial cell. In other embodiments, the microbial cell expresses and secretes the POI into the fermentation broth. Thus, in related embodiments, the above methods further comprise at least one the protein recovery step (process) selected from the group consisting of a cell lysis step, a cell separation step, a protein concentration step and a protein purification step. In another embodiment, one or more of the proteins comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17 are secreted into the fermentation broth. In other embodiments, one or more of the proteins comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17 are retained inside (intracellular) the microbial cell. In a related embodiment, the methods further comprise at least one the protein recovery step selected from the group consisting of a cell lysis step, a cell separation step, a protein concentration step and a protein purification step.

In other embodiments, the disclosure relates to methods for recovering a protein of interest (POI) from a fermentation broth essentially free from contaminating DNA comprising (a) obtaining a microbial cell fermentation broth comprising a protein of interest (POI), (b) treating the broth with one or more exogenously introduced proteins comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17, and (c) recovering the POI from the broth, wherein the recovered POI is essentially free from contaminating DNA. In certain of these embodiments, recovering the POI from the broth comprises at least one protein recovery step selected from the group consisting of a cell lysis step, a cell separation step, a protein concentration step and a protein purification. In another embodiment, at least one protein recovery step is performed in the presence of one or more exogenously introduced proteins comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17. In a preferred embodiment, one or more exogenously introduced proteins are protease resistant.

Thus, certain other embodiments are directed to protein preparations essentially free from contaminating DNA recovered according to the methods of the disclosure. Certain other embodiments are therefore related to isolated proteins of interest essentially free from contaminating DNA recovered according to the methods of the disclosure.

In other embodiments, the disclosure is related to methods for reducing the DNA content of a fermentation broth in which microbial host cells have been fermented, wherein the method comprises introducing into the fermentation broth one or more exogenous proteins comprising at least 85% identity to a protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17. In related embodiments, the broth is subjected to at least one the protein recovery step performed in the presence of the one or more exogenously introduced proteins. In certain of these embodiments, the at least one the protein recovery step is selected from the group consisting of a cell lysis step, a cell separation step, a protein concentration step and a protein purification. In a preferred embodiment, one or more exogenously introduced proteins are protease resistant.

As set forth and described herein, suitable microbial (host) cells for use in the compositions and methods of the disclosure include, but are not limited to, Gram negative bacterial cells, Gram positive bacterial cells, filamentous fungal cells and yeast cells.

### BRIEF DESCRIPTION OF DRAWINGS

FIG. 1 presents the sequences of the prokaryotic and eukaryotic nucleases expressed in the recombinant microbial cells of the disclosure. More specifically, FIG. 1A shows a Thermobifida cellulosilytica nucleic acid sequence (SEQ ID NO: 1) encoding a nuclease named TceNuc1 (SEQ ID NO: 2) and a Trichoderma reesei nucleic acid sequence (SEQ ID NO: 3) encoding a nuclease named TreNuc1 (SEQ ID NO: 4), wherein T. cellulosilytica DNA sequence (SEQ ID NO: 1) and the T. reesei DNA sequence (SEQ ID NO: 3) as presented in FIG. 1A have been codon optimized for expression in Bacillus sp. cells. FIG. 1B shows a Blastomyces gilchristii nucleic acid sequence (SEQ ID NO: 5) encoding a nuclease named BdeNuc1 (SEQ ID NO: 6) and a Gelasinospora tetrasperma nucleic acid sequence (SEQ ID NO: 7) encoding a nuclease named GteNuc1 (SEQ ID NO: 8), wherein the B. gilchristii DNA sequence (SEQ ID NO: 5) and the G. tetrasperma DNA sequence (SEQ ID NO: 7) as presented in FIG. 1B have been codon optimized for expression in Bacillus sp. cells. FIG. 1C shows a Tolypocladium inflatum nucleic acid sequence (SEQ ID NO: 9) encoding a nuclease named TinNuc1 (SEQ ID NO: 10) and a Streptococcus dysgalactiae nucleic acid sequence (SEQ ID NO: 11) encoding a nuclease named SdyNuc1 (SEQ ID NO: 12), wherein the T. inflatum DNA sequence (SEQ ID NO: 9) and the S. dysgalactiae DNA sequence (SEQ ID NO: 11) as presented in FIG. 1C have been codon optimized for expression in Bacillus sp. cells.

FIG. 2 shows an agarose gel image of end of fermentation supernatants of the parental (CB455) strain and modified strains expressing prokaryotic nucleases (TceNuc1 or SdyNuc1). More particularly, as shown in FIG. 2 (from left to right), lanes 1 and 2 are the parental Bacillus strain (CB455), lanes 3-5 are the modified Bacillus strain (CB465) expressing nuclease TceNuc1, lanes 6-8 are the modified Bacillus strain (CB467) expressing nuclease SdyNuc1, and shown in lane M are molecular weight markers (Thermo Scientific™ O'GeneRuler 1 kb DNA Ladder).

FIG. 3 shows an agarose gel image of end of fermentation supernatants of the parental (CB455) strain and modified strains expressing eukaryotic nucleases (TreNuc1, BdeNuc1, GteNuc1 or TinNuc1). More particularly, as shown in FIG. 3 (from left to right), lanes 1-3 are the modified Bacillus strain (CB472) expressing nuclease TreNuc1, lanes 4-6 are the modified Bacillus strain (CB473) expressing nuclease BdeNuc1, lanes 7-9 are the modified Bacillus strain (CB474) expressing nuclease GteNuc1, lanes 10 and 11 are the modified Bacillus strain (CB475) expressing nuclease TinNuc1 and lane 12 is the parental Bacillus strain (CB455) expressing the FNA protease.

FIG. 4 shows schematic diagrams of the TreNuc1 and TinNuc1 nuclease expression cassettes constructed for Trichoderma expression, wherein FIG. 4 (top schematic) shows a diagram for nuclease cassette TreNuc1 (SEQ ID NO: 13), and FIG. 4 (bottom schematic) shows a diagram for nuclease cassette TinNuc1 (SEQ ID NO: 14). As presented in FIG. 4, both the TreNuc1 (SEQ ID NO: 13) and TinNuc1 (SEQ ID NO: 14) cassettes begin with an approximately 1.5 kb region corresponding to the promoter sequence of Trichoderma cbh1, followed by the full coding portion of the nuclease genes, including native introns, from the start codon to the stop codon. The mRNA and coding structures are illustrated (FIG. 4) below the genes with box arrows representing the exons and lines representing the introns. These are followed by an approximately 300 bp region of the Trichoderma cbh1 transcriptional terminator sequence. Following the terminator sequence, is an approximately 2 kb region corresponding to the native Trichoderma pyr2 gene for use as a transformation selection marker. As presented in FIG. 4, the numbering above the diagrams correspond to base pair numbers.

FIG. 5 shows SDS-PAGE analysis of filtrates from microtiter plate fermentations of Trichoderma strains expressing nucleases and control strains. More particularly, as shown in FIG. 5, lanes 1 and 14 are Invitrogen SeeBlue Plus 2 molecular weight marker, with approximate molecular weights on the far right of the figure. Lanes 2, 7, 8 and 13 are pyrimidine prototrophic derivates of the same parental strain as the nuclease transformants, showing the native background proteins expressed by the Trichoderma host. Lanes 3-6 are samples from four (4) different Trichoderma transformants expressing nuclease TreNuc1, and lanes 9-12 are samples from four (4) different Trichoderma transformants expressing nuclease TinNuc1.

FIG. 6 shows SDS-PAGE analysis of bioreactor fermentation samples across the time course of the fermentation, as numbered above lanes, from 47 hours to 186 hours. Samples from a strain expressing nuclease TreNuc1 are on the upper gel (FIG. 6) and samples from a strain expressing nuclease TinNuc1 are on the lower gel (FIG. 6), wherein Invitrogen SeeBlue Plus 2 molecular weight marker as loaded on the far left of the gel and corresponding approximate molecular weights are given on the left of the figure.

FIG. 7 shows an image of an agarose gel electrophoresis of reactions demonstrating nuclease activity in the Trichoderma fermentation broths expressing nucleases. Reaction sample numbers and pertinent components are listed above the lanes. As presented in FIG. 7, the upper half of the gel shows samples incubated at 4° C. for four (4) hours and the bottom gel shows samples incubated at 24° C. for four (4) hours. Invitrogen 1 Kb molecular weight marker was loaded into the center well.

FIG. 8 presents the coding sequence (CDS; SEQ ID NO: 15) of the full-length TinNuc1 nuclease (SEQ ID NO: 16) and the mature TinNuc1 nuclease (SEQ ID NO: 17) expressed in Trichoderma. As shown in FIG. 8, the amino acid residues of the exemplary signal sequence have been underlined (SEQ ID NO: 16).

FIG. 9 shows an agarose gel image of the unformulated (UN; lanes 1-3) and formulated (F; lanes 5-8) polyesterase samples described in TABLE 6, before and after treatment with the TreNuc1 nuclease UFC. As presented in FIG. 9, lanes 1-3 are the unformulated (UN) polyesterase samples, incubated at 4° C.; wherein lane 1 is the untreated sample; lane 2 is the sample treated with 0.1% of TreNuc1 UFC and lane 3 is the sample treated with 1% of TreNuc1 UFC. As presented in FIG. 9, lanes 4-6 are the formulated (F) polyesterase samples, incubated at 4° C.; wherein lane 4 is the untreated sample, lane 5 is the sample treated with 0.1% of TreNuc1 UFC and lane 6 is the sample treated with 1% of TreNuc1 UFC. As shown in FIG. 9, lanes 7 and 8 are the formulated polyesterase samples, incubated at 25° C.; wherein lane 7 is the sample treated with 0.1% of TreNuc1 UFC and lane 8 is the sample treated with 1% of TreNuc1 UFC.

FIG. 10 shows an agarose gel image of the DNA fragments amplified by PCR using oligonucleotides that amplify a specific sequence within the polyesterase gene, using the polyesterase samples described in TABLE 6. As presented in FIG. 10, lane L is a Thermo Scientific™ O'GeneRuler 1 kb DNA Ladder, and lanes 1-8 show the results of the PCR amplification on the untreated samples (lanes 1 and 4) and the samples treated with TreNuc1 UFC (lanes 2, 3 and 5-8) as described in Example 4, TABLE 6.

### BRIEF DESCRIPTION OF THE BIOLOGICAL SEQUENCES

SEQ ID NO: 1 is a Thermobifida cellulosilytica nucleic acid sequence encoding a nuclease named TceNuc1, wherein SEQ ID NO: 1 has been codon optimized for expression in Bacillus sp. cells.

SEQ ID NO: 2 is the amino acid sequence of the TceNuc1 nuclease encoded by SEQ ID NO: 1.

SEQ ID NO: 3 is a Trichoderma reesei nucleic acid sequence encoding a nuclease named TreNuc1, wherein SEQ ID NO: 3 has been codon optimized for expression in Bacillus sp. cells.

SEQ ID NO: 4 is the amino acid sequence of the TreNuc1 nuclease encoded by SEQ ID NO: 3.

SEQ ID NO: 5 is a Blastomyces gilchristii nucleic acid sequence encoding a nuclease named BdeNuc1, wherein SEQ ID NO: 5 has been codon optimized for expression in Bacillus sp. cells.

SEQ ID NO: 6 is the amino acid sequence of the BdeNuc1 nuclease encoded by SEQ ID NO: 5.

SEQ ID NO: 7 is a Gelasinospora tetrasperma nucleic acid sequence encoding a nuclease named GteNuc1, wherein SEQ ID NO: 7 has been codon optimized for expression in Bacillus sp. cells.

SEQ ID NO: 8 is the amino acid sequence of the GteNuc1 nuclease encoded by SEQ ID NO: 7.

SEQ ID NO: 9 is a Tolypocladium inflatum nucleic acid sequence encoding a nuclease named TinNuc1, wherein SEQ ID NO: 9 has been codon optimized for expression in Bacillus sp. cells.

SEQ ID NO: 10 is the amino acid sequence of the TinNuc1 nuclease encoded by SEQ ID NO: 9.

SEQ ID NO: 11 is a Streptococcus dysgalactiae nucleic acid sequence encoding a nuclease named SdyNuc1, wherein SEQ ID NO: 11 has been codon optimized for expression in Bacillus sp. cells.

SEQ ID NO: 12 is the amino acid sequence of the SdyNuc1 nuclease encoded by SEQ ID NO: 11.

SEQ ID NO: 13 is a Trichoderma expression cassette encoding the TreNuc1 nuclease of SEQ ID NO: 4.

SEQ ID NO: 14 is a Trichoderma expression cassette encoding the full length TinNuc1 nuclease of SEQ ID NO: 16.

SEQ ID NO: 15 is the coding sequence of the Trichoderma expression cassette (SEQ ID NO: 14), wherein the cassette encodes the full length TinNuc1 nuclease of SEQ ID NO: 16.

SEQ ID NO: 16 is the predicted amino acid sequence of the full length TinNuc1 nuclease encoded by SEQ ID NO: 15.

SEQ ID NO: 17 is the predicted amino acid sequence of the mature TinNuc1 nuclease encoded by SEQ ID NO: 15.

### DETAILED DESCRIPTION

### I. Overview

As generally described above, the presence of contaminating DNA (e.g., genomic DNA, recombinant DNA) in microbial cell fermentation broths and/or its presence in any down-stream protein recovery processes (steps) thereof, can lead to undesirable protein product qualities. For example, the presence of contaminating DNA in a protein product, such as a protein biologic, an animal feed protein, or a human food additive protein, would require further protein purification steps to remove any residual contaminating DNA. In other scenarios, such as a whole broth product comprising one or more microbial produced enzymes (e.g., a protein preparation), or a multi-enzyme product recovered therefrom, the contaminating DNA may cause broth viscosity problems, and/or interfere with subsequent protein recovery steps, and/or contaminate the final protein product(s), and the like.

As set forth and described hereinafter, certain embodiments of the disclosure, among other things, are related to the identification, isolation and characterization of novel genes encoding novel proteins having nuclease activity, recombinant microbial cells producing various protein products (e.g., proteins of interest) essentially free from DNA, compositions and methods for constructing such recombinant (genetically modified) microbial host cells, compositions and methods of producing and recovering various protein products essentially free from DNA, compositions and methods for rendering protein preparations essentially free from DNA and the like.

### II. Definitions

In view of the novel genes, polynucleotides, coding sequences, open reading frames and the like encoding novel proteins having nuclease activity, and/or the microbial host cells expressing/producing heterologous proteins having nuclease activity, and/or the compositions and methods thereof for mitigating contaminating DNA described herein, the following terms and phrases are defined. Terms not defined herein should be accorded their ordinary meaning as used in the art.

Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described. All publications and patents cited herein are incorporated by reference in their entirety.

It is further noted that the claims may be drafted to exclude any optional element. As such, this statement is intended to serve as antecedent basis for use of such exclusive terminology as “solely,” “only”, “excluding”, “not including” and the like, in connection with the recitation of claim elements, or use of a “negative” limitation or proviso thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, a “microbial cell”, a “microbial host cell”, a “microbial strain” and the like refer to microbial cells that have the capacity to act as a host or expression vehicle for a newly introduced (heterologous) DNA sequence. In certain embodiments, a microbial host cell is selected from the group consisting of a Gram negative bacterial cell, a Gram positive bacterial cell, a filamentous fungal cell and a yeast cell (e.g., recombinant Bacilli cells, recombinant E. coli cells, recombinant fungal cells, recombinant yeast cells).

As used herein, the terms “Gram negative bacteria”, “Gram negative bacterial strains” and “Gram negative bacterial cells” are the same meaning of the terms used in the art. Gram negative bacteria include all bacteria of the classes of proteobacteria, such as alpha-proteobacteria, beta-proteobacteria, gamma-proteobacteria, delta-proteobacteria and epsilon-proteobacteria.

As used herein, the terms “Gram positive bacteria”, Gram positive bacterial strains” and “Gram positive bacterial cells” are the same meaning of the terms used in the art. For example, Gram positive bacteria include all strains of Actinobacteria and Firmicutes. In certain embodiments, such Gram positive bacteria are of the classes Bacilli, Clostridia and Mollicutes.

As used herein, “yeast cells”, “yeast strains”, or simply “yeast” refer to organisms from the phyla Ascomycota and Basidiomycota. Exemplary yeast is budding yeast from the order Saccharomycetales.

As used herein, the term “Ascomycete fungal cell” refers to any organism in the Division Ascomycota in the Kingdom Fungi. Examples of Ascomycetes fungal cells include, but are not limited to, filamentous fungi in the subphylum Pezizomycotina, such as Trichoderma sp., Aspergillus sp., Myceliophthora sp., Penicillium sp., and the like.

As used herein, the terms “filamentous fungus”, “filamentous fungal strains” and “filamentous fungal cells” refers to all filamentous forms of the subdivision Eumycota and Oomycota. For example, filamentous fungi include, without limitation, Acremonium, Aspergillus, Emericella, Fusarium, Humicola, Mucor, Myceliophthora, Neurospora, Penicillium, Scytalidium, Thielavia, Tolypocladium, and Trichoderma species.

As used herein, the terms “recombinant” or “non-natural” refer to an organism, microorganism, cell, nucleic acid molecule, or vector that has at least one engineered genetic alteration, or has been modified by the introduction of a heterologous nucleic acid molecule, or refer to a cell (e.g., a microbial cell) that has been altered such that the expression of a heterologous or endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions or other functional alteration of a cell's genetic material. For example, recombinant cells may express genes or other nucleic acid molecules that are not found in identical or homologous form within a native (wild-type) cell (e.g., a fusion or chimeric protein), or may provide an altered expression pattern of endogenous genes, such as being over-expressed, under-expressed, minimally expressed, or not expressed at all.

As used herein, an exemplary protease named “FNA” is a subtilisin variant (Y217L) derived from the BPN′ (subtilisin) protease of B. amyloliquefaciens. For example, PCT Publication No. WO2011/072099 generally discloses the amino acid sequence of the BPN′ protease and methods for constructing BPN′ variants thereof (e.g., FNA variant Y217L).

As used herein, the product named “Primafast® 200” is a Trichoderma whole cellulase product.

As used herein, an exemplary lipase named “Pseudomonas sp. lipase” was expressed in modified microbial cells of the disclosure (Example 4). For example, PCT Publication No. WO2003/076580 (incorporated herein by referenced in its entirety) generally describes the wild-type Pseudomonas sp. lipase (cutinase) and the construction of variant lipases thereof.

As used herein, a parental B. subtilis strain named “CB455” comprises an expression cassette encoding the FNA protease.

As used herein, the terms “deoxyribonuclease” (abbreviated “DNase”) and “nuclease” may be used interchangeably, and refer to a protein (i.e., enzyme) capable of degrading DNA by (non-specifically) cleaving (hydrolyzing) a phosphodiester bond in the DNA backbone. DNase proteins (enzymes) of the instant disclosure are further defined as non-specific DNases, in contrast to nucleotide sequence-specific DNases, such as restriction enzymes.

For purposes of the present disclosure, the phrases “DNase activity” and a “protein having DNase activity” maybe used interchangeably when referring a protein capable of degrading DNA, such as a protein (e.g., a DNase) which can cut/cleave (degrade) DNA to form oligonucleotides and/or mononucleotides.

For example, in certain embodiments of the disclosure, a protein having DNase activity can cut/cleave (degrade) high-molecular weight DNA into low-molecular weight oligonucleotides and/or mononucleotides thereof. Accordingly, DNA degrading (DNase) activity can be determined according to any of the methods described in Examples section, and/or determined using other suitable DNase methods/assays known in the art. For example, in certain embodiments, a protein having DNase activity comprises an amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 16 or SEQ ID NO: 17, or a protein having DNase activity comprises an amino acid sequence having at least about 60%, at least 70%, at least 80%, at least 90%, or at least 91%-99% sequence identity to a protein of SEQ ID NO: 2, SEQ ID NO: 4, SEQ ID NO: 6, SEQ ID NO: 8, SEQ ID NO: 10 or SEQ ID NO: 12. For example, in certain embodiments, a DNase protein of the disclosure comprises about 95-99% sequence identity to a protein of SEQ ID NO: 2, SEQ ID NO: 4, SEQ ID NO: 6, SEQ ID NO: 8, SEQ ID NO: 10 or SEQ ID NO: 12, provided that the DNase protein does not comprise 100% sequence identity to any one of the amino acid sequences SEQ ID NO: 2, SEQ ID NO: 4, SEQ ID NO: 6, SEQ ID NO: 8, SEQ ID NO: 10 or SEQ ID NO: 12 (i.e., the DNase amino acid sequence does not occur naturally).

As used herein, one (1) unit of DNase activity may be defined as the quantity of enzyme that completely degrades 1 μg of plasmid DNA or high-molecular weight DNA in one (1) hour at 37° C.

As used herein, the DNase named “TceNuc1” is a Thermobifida cellulosilytica nuclease comprising a mature amino acid sequence of SEQ ID NO: 2.

As used herein, the DNase named “TreNuc1” is a Trichoderma reesei nuclease comprising a mature amino acid sequence of SEQ ID NO: 4.

As used herein, the DNase named “BdeNuc1” is a Blastomyces gilchristii nuclease comprising a mature amino acid sequence of SEQ ID NO: 6.

As used herein, the DNase named “GteNuc1” is a Gelasinospora tetrasperma nuclease comprising a mature amino acid sequence of SEQ ID NO: 8.

As used herein, the DNase named “TinNuc1” is a Tolypocladium inflatum nuclease comprising a mature amino acid sequence of SEQ ID NO: 10.

As used herein, the DNase named “SdyNuc1” is a Streptococcus dysgalactiae nuclease comprising a mature amino acid sequence of SEQ ID NO: 12.

As used herein, a modified B. subtilis strain named “CB465” was derived from parental strain CB455, wherein the modified CB465 strain expresses the heterologous T. cellulosilytica DNase TceNuc1 (SEQ ID NO: 2); a modified B. subtilis strain named “CB472” was derived from parental strain CB455, wherein the modified CB472 strain expresses the heterologous T. reesei DNase TreNuc1 (SEQ ID NO: 4); a modified B. subtilis strain named “CB473” was derived from parental strain CB455, wherein the modified CB473 strain expresses the heterologous B. gilchristii DNase BdeNuc1 (SEQ ID NO: 6); a modified B. subtilis strain named “CB474” was derived from parental strain CB455, wherein the modified CB474 strain expresses the heterologous G. tetrasperma DNase GteNuc1 (SEQ ID NO: 8); a modified B. subtilis strain named “CB475” was derived from parental strain CB455, wherein the modified CB475 strain expresses the heterologous T. inflatum DNase TinNuc1 (SEQ ID NO: 10); and a modified B. subtilis strain named “CB467” was derived from parental strain CB455, wherein the modified CB467 strain expresses the heterologous S. dysgalactiae DNase SdyNuc1 (SEQ ID NO: 12).

The phrase “animal feed enzyme”, as used herein includes, but is not limited to, enzymes fed or administered to non-human animals (e.g., cows, birds, chickens, pigs, etc.) such as phytases, proteases, cellulases, β-glucanases, xylanases, lipases, mannanases, α-galactosidases, pectinases, amylases, and the like.

The phrase “food enzyme” as used herein includes, but is not limited to, enzymes which may be added to food ingredients (e.g., dairy products, starches/carbohydrates, fats/lipids, proteins preparations, beer, beverages, etc.) such as lactases, amylases, proteases, cellulases, lipases, xylanases and the like

As defined herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, ultrafiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, a “protein preparation” is any material, typically a solution, generally aqueous, comprising one or more proteins.

As used herein, the terms “broth”, “cultivation broth” and “fermentation broth” may be used interchangeably, and particularly refer to a whole fermentation broth.

The term “whole fermentation broth” as used herein refers to a preparation produced by cellular fermentation that undergoes no or minimal recovery and/or purification. For example, whole fermentation broths are produced when microbial cultures are grown to saturation, incubated under carbon-limiting conditions to allow protein synthesis (e.g., expression of proteins by host cells) and secretion into cell culture medium. Typically, the whole fermentation broth is unfractionated and comprises spent cell culture medium, extracellular polypeptides, and microbial cells.

“Cell debris” refers to cell walls and other insoluble cellular components that are released after disruption of the cell membrane, e.g., after lysis of microbial cells.

“Cell kill” means a process rendering the host organisms inactive and no longer able to replicate.

“Broth conditioning” refers to pretreatment of a microbial fermentation broth designed to improve subsequent broth handling properties. Broth conditioning changes the chemical composition and/or physical and/or rheological properties of the broth to facilitate its use in downstream recovery and/or formulation processes. Broth conditioning may include one or more treatments such as pH modification, heat treatment, cooling, addition of additives (e.g., calcium, salt(s), flocculant(s), reducing agent(s), enzyme activator(s), enzyme inhibitor(s), and/or surfactant(s)), mixing, and/or timed hold (e.g., 0.5 to 200 hours) of the broth without further treatment. As set forth and further described below, certain aspects of the disclosure provide novel DNase compositions (e.g., DNase protein preparations) suitable for degrading contaminating DNA present in microbial cell fermentation broths. In related embodiments, a DNase preparation of the disclosure is added during a broth conditioning/treatment process, including but not limited to, broth stabilization processes, broth pH and/or temperature optimization processes, broth conditioning with additives, broth holding times, and the like.

The terms “recovery”, “recovered” and “recovering” as used herein refer to treatment or stabilization of broth, or at least partial separation of a protein from one or more soluble components of a microbial broth and/or at least partial separation from one or more solvents in the broth (e.g., water or ethanol). A recovered protein is often of higher purity than prior to the recovery process. However, in some embodiments, a recovered protein may be of the same or lower purity than prior to the recovery process.

As used herein, when the expression/production of a DNase and/or the expression/production of a protein of interest (POI) in an “unmodified” (parental or control) cell is being compared to the expression/production of the same DNase and/or POI in a “modified” (recombinant daughter) cell, it will be understood that the “modified” and “unmodified” cells are grown/cultivated/fermented under the same conditions (e.g., the same conditions such as media, temperature, pH and the like). Thus, a DNase and/or a POI of the disclosure may be produced inside the host cell, or secreted (or transported) into the culture medium.

As used herein, the terms “modification” and “genetic modification” are used interchangeably and include: (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) the down-regulation of a gene, (f) specific mutagenesis and/or (g) random mutagenesis of any one or more the genes disclosed herein.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any step involved in the production of the polypeptide including, but not limited to, transcription, post-transcriptional modification, translation, post-translational modification, secretion and the like.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein. It is understood that the polynucleotides (or nucleic acid molecules) described herein include “genes”, “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence.

As used herein, the term “coding sequence” refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” as used herein refers to the association of nucleic acid sequences on a single nucleic acid fragment so that the function of one is affected by the other. For example, a promoter is operably linked with a coding sequence (e.g., an ORF) when it is capable of affecting the expression of that coding sequence (i.e., that the coding sequence is under the transcriptional control of the promoter). Coding sequences can be operably linked to regulatory sequences in sense or antisense orientation.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in the microbial cell of interest. For example, in certain embodiments, the present disclosure is directed to a polynucleotide comprising a 5′ promoter (or 5′ promoter region, or tandem 5′ promoters and the like), wherein the promoter region is operably linked to a nucleic acid sequence (e.g., an ORF) encoding a protein.

As used herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure.

As used herein, the term “introducing”, as used in phrases such as “introducing into a cell” or “introducing into a (microbial) cell at least one polynucleotide open reading frame (ORF), or a gene thereof, or a vector thereof, includes methods known in the art for introducing polynucleotides into a cell, including, but not limited to protoplast fusion, natural or artificial transformation (e.g., calcium chloride, electroporation), transduction, transfection, conjugation and the like.

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). Transformation therefore generally refers to introducing an exogenous DNA into a host cell so that the DNA is maintained as a chromosomal integrant or a self-replicating extra-chromosomal vector.

As used herein, “disruption of a gene” or a “gene disruption”, are used interchangeably and refer broadly to any genetic modification that substantially prevents a host cell from producing a functional gene product (e.g., a protein). Thus, as used herein, a gene disruption includes, but is not limited to, frameshift mutations, premature stop codons (i.e., such that a functional protein is not made), substitutions eliminating or reducing activity of the protein internal deletions (such that a functional protein is not made), insertions disrupting the coding sequence, mutations removing the operable link between a native promoter required for transcription and the open reading frame, and the like.

As used herein “an incoming sequence” refers to a DNA sequence that is introduced into the microbial cell. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, the nonfunctional sequence may be inserted into a gene to disrupt function of the gene. In another embodiment, the incoming sequence includes a selective marker. In a further embodiment the incoming sequence includes two homology boxes.

As used herein, “homology box” refers to a nucleic acid sequence, which is homologous to a sequence in the microbial cell. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, down-regulated and the like, according to the invention. These sequences direct where in the chromosome a DNA construct is integrated and directs what part of the chromosome is replaced by the incoming sequence. While not meant to limit the present disclosure, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. The sequence of each homology box is homologous to a sequence in the chromosome. These sequences direct where in the chromosome the new construct gets integrated and what part of the chromosome will be replaced by the incoming sequence. In other embodiments, the 5′ and 3′ ends of a selective marker are flanked by a polynucleotide sequence comprising a section of the inactivating chromosomal segment. In some embodiments, a flanking sequence is present on only a single side (either 3′ or 5′), while in other embodiments, it is present on each side of the sequence being flanked.

As used herein, the terms “selectable marker” and “selective marker” refer to a nucleic acid (e.g., a gene) capable of expression in host cell which allows for ease of selection of those hosts containing the vector. Examples of such selectable markers include, but are not limited to, antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell. in some embodiments plasmids exist in a parental cell and are lost in the daughter cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously or can integrate into a chromosome of a host organism).

An “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector.

As used herein, the term “protein of interest” or “POI” refers to a polypeptide of interest that is desired to be expressed in a recombinant microbial cell of the disclosure. Thus, as used herein, a POI may be an enzyme, a substrate-binding protein, a surface-active protein, a structural protein, a receptor protein,

Similarly, as defined herein, a “gene of interest” or “GOI” refers a nucleic acid sequence (e.g., a polynucleotide, a gene or an ORF) which encodes a POI. A “gene of interest” encoding a “protein of interest” may be a naturally occurring gene, a mutated gene or a synthetic gene.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In certain embodiments, a gene of the instant disclosure encodes a commercially relevant industrial protein of interest, such as an enzyme (e.g., a acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, lysozymes, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polyesterases, polygalacturonases, proteases, peptidases, rhamno-galacturonases, ribonucleases, transferases, transport proteins, transglutaminases, xylanases, hexose oxidases, and combinations thereof).

A “variant” of an enzyme, protein, polypeptide, nucleic acid, or polynucleotide, as used herein means that the variant is derived from a parent polypeptide or parent nucleic acid (e.g., native, wildtype or other defined parent polypeptide or nucleic acid) that includes at least one modification or alteration as compared to that parent. Thus, a variant may have a few mutations as compared to a parent, where by “a few” is meant from 1 to 10 mutations. For example, a variant having from 1 to 10 amino acid substitutions as compared to a DNase protein of SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17, can be referred to as a DNase variant having a few substitutions. Such alterations/modifications can include a substitution of an amino acid/nucleic acid residue in the parent for a different amino acid/nucleic acid residue at one or more sites, deletion of an amino acid/nucleic acid residue (or a series of amino acid/nucleic acid residues) in the parent at one or more sites, insertion of an amino acid/nucleic acid residue (or a series of amino acid/nucleic acid residues) in the parent at one or more sites, truncation of amino-terminal amino acid and/or carboxy-terminal amino acid sequences or 5′ and or 3′ nucleic acid sequences, and any combination thereof.

A variant DNase protein according to aspects of the present disclosure retains DNase (enzyme) activity, but may have an altered property in some other specific aspects (e.g., an improved property). For example, a variant DNase protein (enzyme) of the disclosure may have an altered pH optimum, improved thermostability or oxidative stability, or a combination thereof, but will retain its characteristic DNase (enzyme) activity. In some embodiments, a variant DNase comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 2, SEQ ID NO: 4, SEQ ID NO: 6, SEQ ID NO: 8, SEQ ID NO: 10, SEQ ID NO:12, SEQ ID NO:16 or SEQ ID NO: 17, or an enzymatically active fragment thereof.

A “parent” or “parental” polynucleotide, polypeptide, or enzyme sequence (e.g., a “parent DNase”), or equivalents thereto, as used herein refers to a polynucleotide, polypeptide, or enzyme sequence that was used as a starting point or template for designing a variant polynucleotide, polypeptide, or enzyme. In certain embodiments, the parent DNase is a DNase protein (enzyme) set forth as SEQ ID NO: 2, SEQ ID NO: 4, SEQ ID NO: 6, SEQ ID NO: 8, SEQ ID NO: 10, SEQ ID NO:12, SEQ ID NO:16 and/or SEQ ID NO: 17.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or a sequence thereof, the term “substitution” means the replacement (i.e., substitution) of one amino acid with another amino acid.

As defined herein, an “endogenous gene” refers to a gene in its natural location in the genome of an organism.

As defined herein, a “heterologous” gene, a “non-endogenous” gene, or a “foreign” gene refer to a gene (or ORF) not normally found in the host organism, but that is introduced into the host organism by gene transfer. As used herein, the term “foreign” gene(s) comprise native genes (or ORFs) inserted into a non-native organism and/or chimeric genes inserted into a native or non-native organism.

As defined herein, a “heterologous control sequence”, refers to a gene expression control sequence (e.g., a promoter or enhancer) which does not function in nature to regulate (control) the expression of the gene of interest. Generally, heterologous nucleic acid sequences are not endogenous (native) to the cell, or a part of the genome in which they are present, and have been added to the cell, by infection, transfection, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “derived” encompasses the terms “originated” “obtained,” “obtainable,” and “created,” and generally indicates that one specified material or composition finds its origin in another specified material or composition, or has features that can be described with reference to another specified material or composition.

As used herein, the term “homology” relates to homologous polynucleotides or polypeptides. If two or more polynucleotides or two or more polypeptides are homologous, this means that the homologous polynucleotides or polypeptides have a “degree of identity” of at least 60%, more preferably at least 70%, even more preferably at least 85%, still more preferably at least 90%, more preferably at least 95%, and most preferably at least 98%. Whether two polynucleotide or polypeptide sequences have a sufficiently high degree of identity to be homologous as defined herein, can suitably be investigated by aligning the two sequences using a computer program known in the art, such as “GAP” provided in the GCG program package (Program Manual for the Wisconsin Package, Version 8, August 1994, Genetics Computer Group, 575 Science Drive, Madison, Wisconsin, USA 53711) (Needleman and Wunsch, (1970). Using GAP with the following settings for DNA sequence comparison: GAP creation penalty of 5.0 and GAP extension penalty of 0.3.

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein, “aerobic fermentation” refers to growth in the presence of oxygen, and “anaerobic fermentation” refers to growth in the absence of oxygen.

### III. Polynucleotides Encoding Proteins Having Dnase Activity

As briefly set forth above, certain embodiments of the disclosure are related to the identification isolation and characterization of novel genes encoding novel proteins having DNase activity. For example, as generally described herein, the DNase proteins (i.e., enzymes) of the disclosure are particularly useful in removing (degrading) contaminating DNA. More specifically, as presented in instant examples, Applicant has constructed recombinant microbial cells expressing these heterologous proteins having DNase activity.

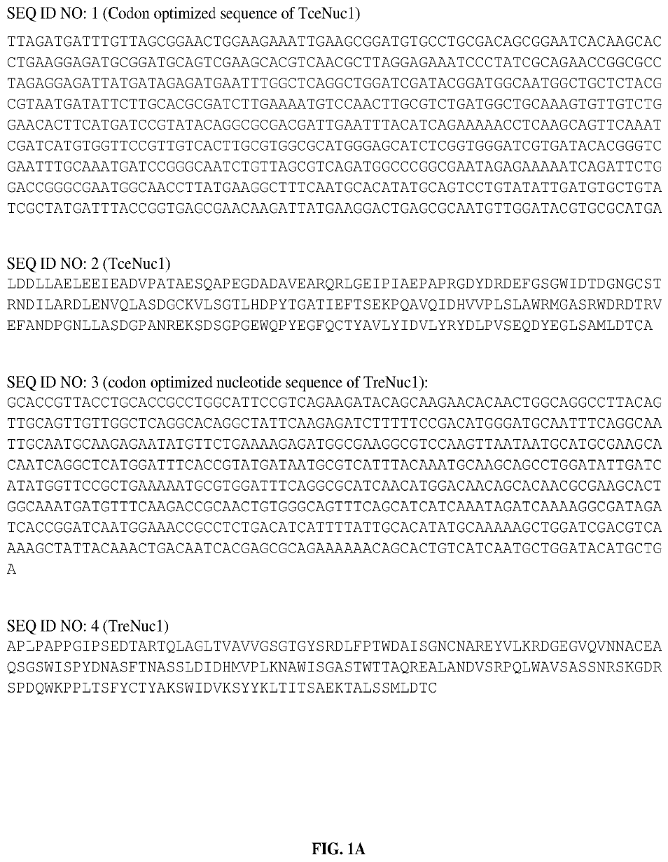

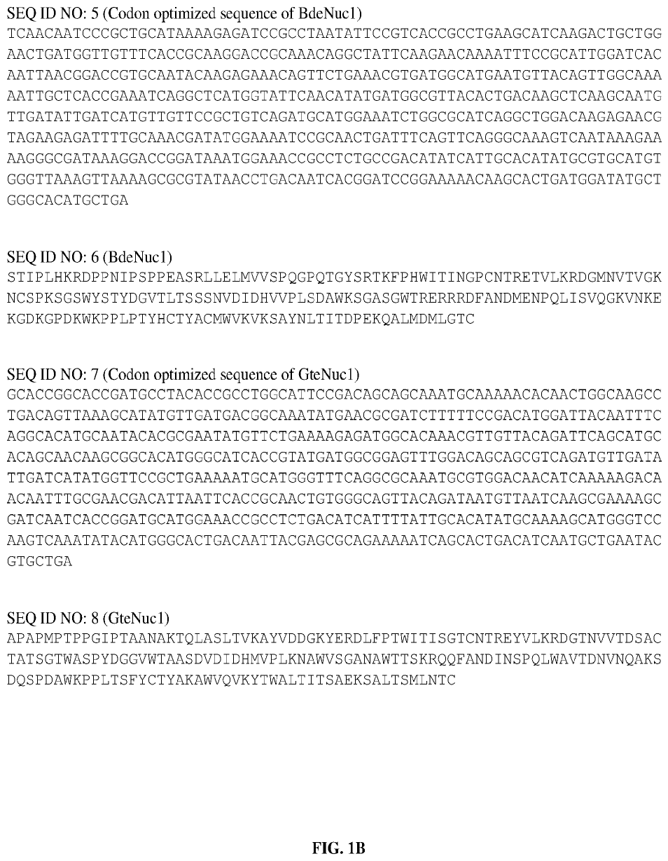

For example, as shown in FIG. 1, a nucleic acid sequence was isolated from Thermobifida cellulosilytica (SEQ ID NO: 1) encoding a mature protein comprising the amino acid sequence of SEQ ID NO: 2 (FIG. 1A; DNase TceNuc1), a nucleic acid sequence was isolated from Trichoderma reesei (SEQ ID NO: 3) encoding a mature protein comprising the amino acid sequence of SEQ ID NO: 4 (FIG. 1A; DNase TreNuc1), a nucleic acid sequence isolated from Blastomyces gilchristii (SEQ ID NO: 5) encoding a mature protein comprising the amino acid sequence of SEQ ID NO: 6 (FIG. 1B; DNase BdeNuc1), a nucleic acid sequence was isolated from Gelasinospora tetrasperma (SEQ ID NO: 7) encoding a mature protein comprising the amino acid sequence of SEQ ID NO: 8 (FIG. 1B; DNase GteNuc1), a nucleic acid sequence was isolated from Tolypocladium inflatum (SEQ ID NO: 9) encoding a mature protein comprising the amino acid sequence of SEQ ID NO: 10 (FIG. 1C; DNase TinNuc1), and a nucleic acid sequence was isolated from Streptococcus dysgalactiae (SEQ ID NO: 11) encoding a mature protein comprising the amino acid sequence of SEQ ID NO: 12 (FIG. 1C; DNase SdyNuc1).

More specifically, as generally described in Examples below, the above prokaryotic proteins (SEQ ID NO: 2 and SEQ ID NO: 12) and eukaryotic proteins (SEQ ID NO: 4, SEQ ID NO: 6, SEQ ID NO: 8 and SEQ ID NO: 10) comprise DNase activity, which DNases are particularly useful for degrading contaminating DNA (e.g., rDNA, gDNA).

For example, in particular embodiments, Applicant has co-expressed the above prokaryotic and eukaryotic DNases with an exemplary enzyme (i.e., FNA protease). As generally described in Example 1, a parental Bacillus subtilis strain (CB455) expressing the FNA protease was modified to co-express a heterologous prokaryotic DNase, such as the modified B. subtilis strain CB465 (co-expressing DNase TceNuc1) and the modified B. subtilis strain CB467 (co-expressing DNase SdyNuc1). More specifically, the parental (CB455) and modified Bacillus strains co-expressing a DNase (TceNuc1 or SdyNuc1) were fermented under identical conditions, wherein quantification of recombinant DNA (rDNA; Example 1, TABLE 2) demonstrates that the prokaryotic DNase TceNuc1 effectively degrades the rDNA present in the supernatant of the cell cultures (e.g., due to cell lysis).

Likewise, as described in Example 2, the parental B. subtilis strain (CB455) expressing the FNA protease was modified to co-express a heterologous eukaryotic DNase, such as modified B. subtilis strain CB472 (co-expressing DNase TreNuc1), the modified strain CB473 (co-expressing DNase BdeNuc1), the modified strain CB474 (co-expressing DNase GteNuc1) and the modified strain CB475 (co-expressing DNase TinNuc1). More specifically, the parental (CB455) and the modified B. subtilis strains co-expressing a DNase (TreNuc1, BdeNuc1, GteNuc1 or TinNuc1) were fermented under identical conditions, wherein the rDNA quantification results (Example 2, TABLE 4), demonstrate that the DNases BdeNuc1, GteNuc1 and TinNuc1 are particularly suitable for degrading residual DNA present in the supernatants of the cultures. As set forth in Examples 1 and 2, it was further observed that all of these DNase proteins (i.e., TceNuc1, SdyNuc1, TreNuc1, BdeNuc1, GteNuc1 and TinNuc1) were surprisingly protease resistant, as expressed in the presence of the FNA (subtilisin) protease.

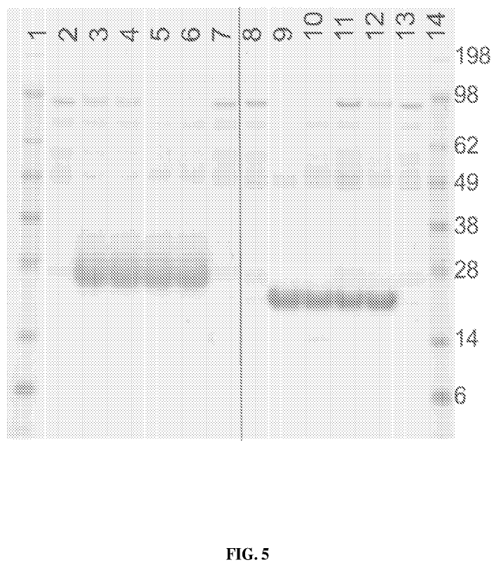

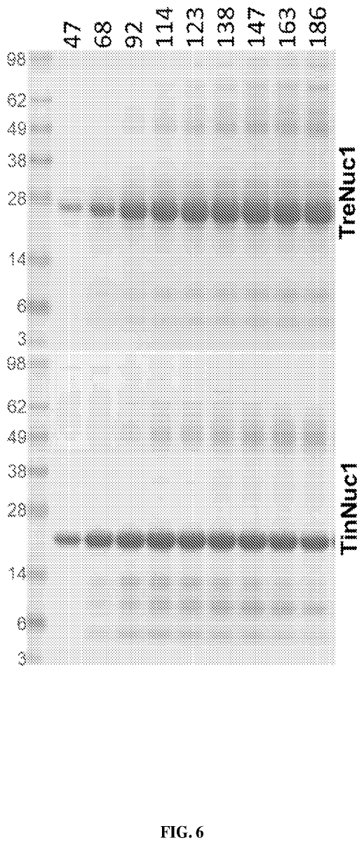

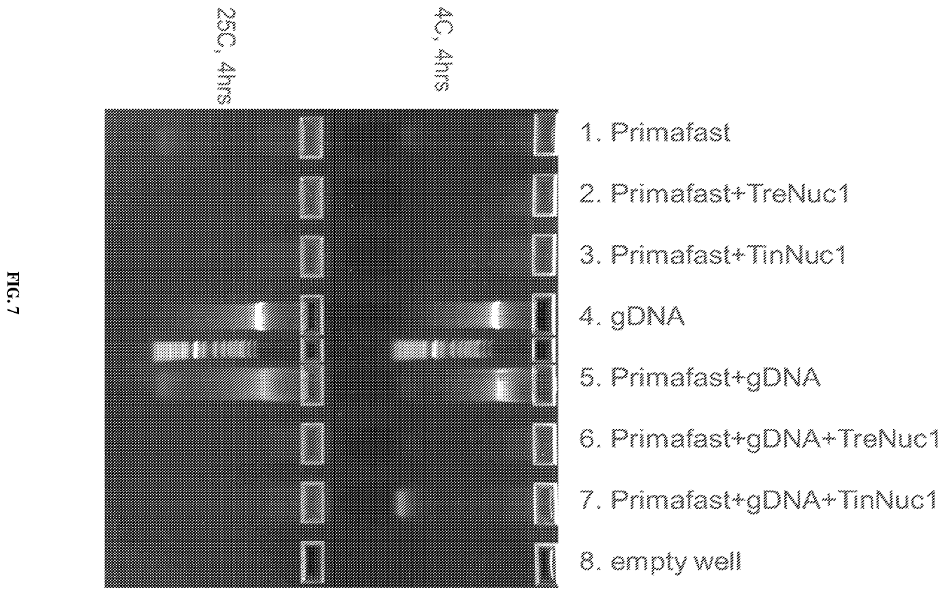

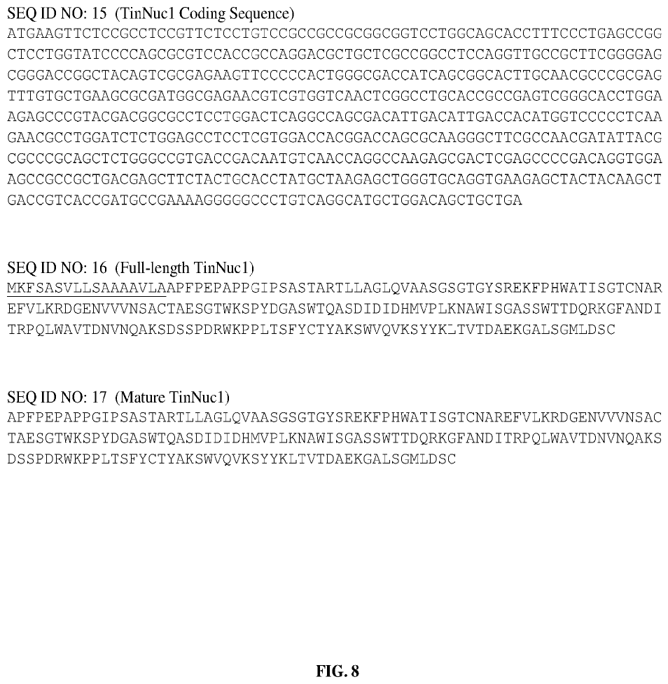

Additionally, as described in Example 3, an exemplary filamentous fungal strain was transformed with expression cassettes encoding the eukaryotic DNases (i.e., DNases TreNuc1 and TinNuc1), wherein the transformed strains were screened for nuclease expression via fermentation in microtiter plates and by fermentation in two (2) L bioreactors. As presented in FIG. 6, both nucleases (TreNuc1 and TinNuc1) expressed well in the bioreactors. As further described in Example 3, the expressed recombinant DNases (TreNuc1 and TinNuc1) can efficiently remove (degrade) DNA from a concentrated Trichoderma whole cellulase product (Primafast® 200). For example, as shown in FIG. 7, with the addition of broth from either the TreNuc1 or TinNuc1 DNase expressing strains, all high molecular weight DNA was eliminated at either incubation temperature tested.

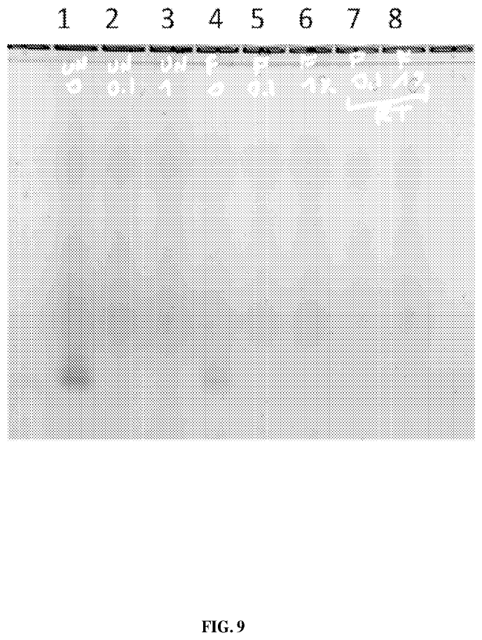

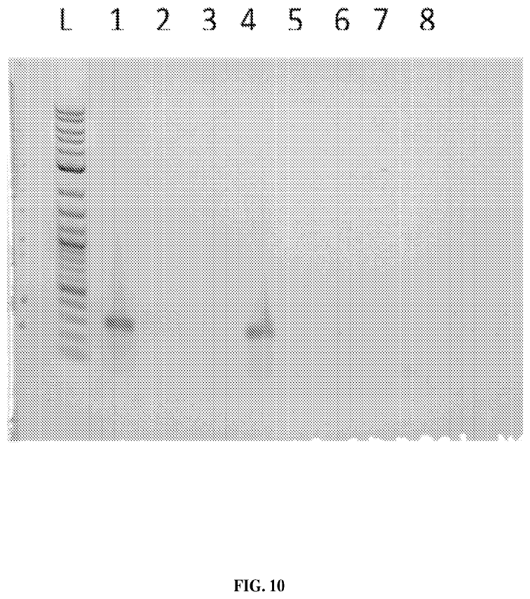

Example 4 of the disclosure generally describes DNase treatments suitable for use on whole fermentation broths, supernatants, protein preparations, isolated proteins, concentrated proteins and the like, using the DNase TreNuc1 as an exemplary DNase. More specifically, a DNase TreNuc1 concentrate (i.e., an ultrafiltration concentrate (UFC) of DNase TreNuc1) was used to treat an ultrafiltration concentrate (UFC) obtained from the fermentation of a B. subtilis strain overexpressing a heterologous Pseudomonas sp. lipase. For example, FIG. 9 shows an agarose gel image of the unformulated (UN) and formulated (F) lipase samples before and after treatment with the DNase TreNuc1 UFC, and FIG. 10 shows an agarose gel image of the DNA fragments amplified by PCR before and after treatment with the DNase TreNuc1 UFC. More particularly, the absence of an amplified PCR product (i.e., contaminating DNA) in the samples treated with the DNase TreNuc1 UFC demonstrates that the DNase activity of the TreNuc1 UFC can effectively degrade any contaminating DNA present in the lipase samples.

Thus, as set forth and contemplated herein, such novel DNase proteins of the disclosure, and/or functional DNase variant proteins derived or obtained therefrom and/or functional fragments thereof (e.g., comprising DNase activity) are particularly useful in mitigating, and removing (degrading) contaminating DNA. In certain aspects, a DNase protein of the disclosure is active in aqueous solutions, and is capable of degrading/cleaving plasmids and high/low molecular weight DNA into low molecular weight oligonucleotides and/or mononucleotides.

Thus, in certain aspects the DNase proteins of SEQ ID NO: 2 and/or SEQ ID NO: 12, are particularly useful and active in aqueous solutions at incubation temperatures between about 25° C. to about 40° C. and pH ranges of about pH 6 to about pH 10. For example, in certain aspects, the DNase proteins of SEQ ID NO: 2 and/or SEQ ID NO: 12 are particularly useful and active in aqueous solutions at incubation temperatures of about 25° C., 26° C., 27° C., 28° C., 29° C., 30° C., 31° C., 32° C., 33° C., 34° C., 35° C., 36° C., 37° C. and 38° C., and pH ranges of about pH 5.5, pH 6, pH 6.5, pH 7, pH 7.5, pH 8 and pH 8.5.

Likewise, the DNase proteins of SEQ ID NO: 4, SEQ ID NO: 6, SEQ ID NO: 8 and/or SEQ ID NO: 10, are particularly useful and active in aqueous solutions at incubation temperatures between about 4° C. to about 40° C. and pH ranges of about pH 5 to about pH 10. For example, in certain aspects, the DNase proteins of SEQ ID NO: 4, SEQ ID NO: 6, SEQ ID NO: 8 and/or SEQ ID NO: 10 are particularly useful and active in aqueous solutions at incubation temperatures of about 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C. and 38° C. and pH ranges of about pH 4.5, pH 5, pH 5.5, pH 6, pH 6.5, pH 7, pH 7.5, pH 8 and pH 8.5.

Additionally, as set forth above, an extended benefit of present DNase proteins is their resistance to protease degradation in such aqueous solutions, incubation temperatures, pH ranges and the like.

Thus, certain aspects of the disclosure are related to DNase proteins comprising an amino acid sequence of SEQ ID NO: 2, SEQ ID NO: 4, SEQ ID NO: 6, SEQ ID NO: 8, SEQ ID NO: 10 or SEQ ID NO: 12 and/or functional fragments thereof (i.e., comprising DNase activity). Certain other aspects are related to variant DNase proteins (e.g., comprising at least 85% to about 99% identity to a parent DNase protein of SEQ ID NO: 2, SEQ ID NO: 4, SEQ ID NO: 6, SEQ ID NO: 8, SEQ ID NO: 10 or SEQ ID NO: 12 and/or functional fragments thereof). For example, in certain embodiments, a variant DNase protein comprises at least 85% identity to a protein of SEQ ID NO: 2, SEQ ID NO: 4, SEQ ID NO: 6, SEQ ID NO: 8, SEQ ID NO: 10 or SEQ ID NO: 12, and comprises at least one modified amino acid residue relative to SEQ ID NO: 2, SEQ ID NO: 4, SEQ ID NO: 6, SEQ ID NO: 8, SEQ ID NO: 10 or SEQ ID NO: 12, respectively. In other embodiments, a variant DNase protein comprises at least 90% to about 99% identity to a DNase protein of SEQ ID NO: 2, SEQ ID NO: 4, SEQ ID NO: 6, SEQ ID NO: 8, SEQ ID NO: 10 or SEQ ID NO: 12, and has at least one modified amino acid residue relative to SEQ ID NO: 2, SEQ ID NO: 4, SEQ ID NO: 6, SEQ ID NO: 8, SEQ ID NO: 10 or SEQ ID NO: 12, respectively.

More particularly, Applicant has analyzed the primary amino acid sequences of the parent DNase proteins set forth above, which computational analysis indicated all of these DNase proteins belong to a large nuclease superfamily called the His-Me finger (alternatively referred to as His-Asn-His (HNH) nucleases and/or ββα-Me nucleases). For example, the HNH family nucleases possess a common nuclease binding and cleavage motif of about 30 to 40 amino acids that contain the three conserved His-Asn-His residues, but share limited amino acid sequence homology. Included in this superfamily are homing endonucleases, colicins, restriction endonucleases, transposases and DNA packaging factors. Although these nucleases function in diverse ways, His-Me fingers fold into similar structures (βμα-metal topology with two antiparallel β-strands, one α-helix, and a divalent metal ion bound in the center), and act as a scissor that binds and cleaves nucleic acids by a conserved one-metal-ion dependent mechanism. A general review of the structures, mechanisms, and functions of the His-Me (HNH) nucleases are described in Wu et al. 2020 (incorporated herein by reference in its entirety).

Thus, as described and contemplated herein, DNase variant proteins of the disclosure are readily envisioned by one skilled in the art, and may be constructed based on the primary amino acid sequences of SEQ ID NO: 2, SEQ ID NO: 4, SEQ ID NO: 6, SEQ ID NO: 8, SEQ ID NO: 10 or SEQ ID NO: 12, along with the HNH (ββα-Me) nuclease domains, including such DNA-binding and cleavage motifs and/or by reference to other members of the HNH superfamily (e.g., homing endonucleases, colicins, restriction endonucleases, transposases and DNA packaging factors).

### IV. Recombinant Nucleic Acids and Molecular Biology

Certain embodiments of the disclosure are directed to isolated nucleic acids (polynucleotides) comprising at least 80% identity to a nucleotide sequence of SEQ ID NO: 1, SEQ ID NO: 3, SEQ ID NO: 5, SEQ ID NO: 7, SEQ ID NO: 9, SEQ ID NO: 11 or SEQ ID NO: 15. For example, in particular embodiments an isolated nucleic acid of the disclosure comprises at least 80% identity to a nucleotide sequence of SEQ ID NO: 1, SEQ ID NO: 3, SEQ ID NO: 5, SEQ ID NO: 7, SEQ ID NO: 9, SEQ ID NO: 11 or SEQ ID NO:15, and encodes a protein having DNase activity. In related embodiments, an isolated nucleic acid comprises at least 80% identity to a nucleotide sequence of SEQ ID NO: 1, SEQ ID NO: 3, SEQ ID NO: 5, SEQ ID NO: 7, SEQ ID NO: 9, SEQ ID NO: 11, or SEQ ID NO: 15 and encodes a protein having DNase activity comprising at least 85% identity to a protein of SEQ ID NO: 2, SEQ ID NO: 4, SEQ ID NO: 6, SEQ ID NO: 8, SEQ ID NO: 10, SEQ ID NO: 12 or SEQ ID NO: 16, respectively. Thus, certain other embodiments are related to plasmids, vectors, expression cassettes and the like comprising a polynucleotide sequence encoding protein of the disclosure comprising DNase activity. Likewise, other embodiments are directed to recombinant microbial cells (strains) expressing one or more heterologous proteins having DNase activity, and in other embodiments, related to a recombinant microbial cells co-expresses (i) a protein of interest (POI) and (ii) one or more proteins having DNase activity.

More particularly, in certain embodiments, a gene, polynucleotide or ORF of the disclosure encoding a protein of interest and/or encoding a protein having DNase activity is genetically modified, e.g., genetic modifications including, but not limited to, (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) the down-regulation of a gene, (f) specific mutagenesis and/or (g) random mutagenesis of any one or more the genes disclosed herein.

Those of skill in the art are well aware of suitable methods for introducing polynucleotide sequences into bacterial cells (e.g., E. coli, Bacillus sp., etc.), filamentous fungal cells (e.g., Aspergillus sp., Trichoderma sp., etc.), yeast cells (e.g., Saccharomyces sp.) and the like (i.e., microbial cells).

As generally specified above, certain embodiments of the disclosure are directed to expressing, producing and/or secreting one or more proteins of interest which are heterologous to the to the microbial host cell. Therefore, the instant disclosure generally relies on routine techniques in the field of recombinant genetics. Basic texts disclosing the general methods of use in present disclosure include Sambrook et al., (1989; 2011; 2012); Kriegler (1990) and Ausubel et al., (1994).

In particular embodiments, the disclosure relates to recombinant (modified) nucleic acids comprising a gene or ORF encoding a DNase protein (e.g., SEQ ID NO: 2, 4, 6, 8, 10, 12, 16 or 17) or variant DNase proteins thereof. For example, in certain embodiments, a recombinant nucleic acid is a polynucleotide expression cassette for expression/production of a DNase protein of the disclosure. In certain other embodiments, the recombinant nucleic acid (polynucleotide) further comprises one or more selectable markers. Selectable markers for use in Gram negative bacteria, Gram positive bacteria filamentous fungi and yeast are generally known in the art. Thus, in certain embodiments, a polynucleotide construct encoding DNase protein, or a POI, comprises a nucleic acid sequence encoding a selectable marker operably linked thereto.

In other embodiments, nucleic acids comprising a gene (or ORF) encoding a DNase protein further comprises operably linked regulatory or control sequences. An example of regulatory or control sequences may be a promoter sequence or a functional part thereof, (i.e., a part which is sufficient for affecting expression of the nucleic acid sequence). Other control sequences include, but are not limited to, a leader sequence, a pro-peptide sequence, a signal sequence, a transcription terminator, a transcriptional activator and the like. Thus, in certain embodiments, a recombinant (modified) polynucleotide comprises an upstream (5′) promoter (pro) sequence driving the expression of a gene (or ORF) encoding a DNase protein, or a POI of the disclosure. More particularly, in certain embodiments, the promoter is a constitutive or an inducible promoter active (functional) in the microbial host cell. For example, one of sill in the art can use any suitable promoter capable of driving the expression of a gene of interest in a microbial expression host cell. Thus, in certain aspects, a recombinant nucleic acid of the disclosure comprises a promoter (pro) sequence which is 5′ (upstream) and operably linked to a nucleic acid (gene) sequence encoding a DNase protein (e.g., 5′-[pro]-[gene]-3′).

In certain other aspects, a recombinant nucleic acid (e.g., an expression cassette) comprises an upstream (5′) promoter (pro) sequence operably linked to a downstream (3′) nucleic acid encoding a DNase protein (or encoding a POI), further comprises a terminator (term) sequence downstream and operably linked thereto. For example, in certain embodiments, a recombinant nucleic acid of the disclosure comprises a promoter (pro) sequence which is 5′ (upstream) and operably linked to a nucleic acid (gene) sequence encoding a DNase protein which is operably linked to a downstream terminator (term) sequence (e.g., 5′-[pro]-[gene]-[term]-3′).

Suitable promoters for driving the expression of genes of interest in a microbial host cell of the disclosure are generally known in the art. For example, exemplary Bacillus sp. promoters include, but are not limited to, tac promoter sequences, β-lactamase promoter sequences, aprE promoter sequences, groES promoter sequences, ftsH promoter sequences, tufA promoter sequences, secDF promoter sequences, minC promoter sequences, spoVG promoter sequences, veg promoter sequences, hbs promoter sequences, amylases promoter sequences, P43 promoter sequence and the like, exemplary filamentous fungal promoters include, but are not limited to, Trichoderma sp. promoters (e.g., cellobiohydrolase promoters, endoglucanase promoters, β-glucosidase promoters, xylanases promoters, glucoamylase promoters), Aspergillus sp. promoters (e.g., trpC promoters, glucoamylase promoters), and the like. However, it is not intended that the present disclosure be limited to any particular promoter, as any suitable promoter known to those in the art finds use with the present invention.

Thus, certain other embodiments are related to cultivating (fermenting) microbial host cells expressing a DNase protein and/or a POI, wherein the expressed DNase and/or POI are secreted into the culture (fermentation) broth. For example, in certain other embodiments, a recombinant nucleic acid comprises an upstream (5′) heterologous promoter (pro) sequence operably linked to a downstream (3′) nucleic acid sequence (ss) encoding a protein signal sequence operably linked to a downstream (3′) nucleic acid (gene) encoding a DNase protein (e.g., 5′-[pro]-[ss]-[gene]-3′).

Any suitable (protein) signal sequence (signal peptide) functional in the microbial cell of choice may be used for the secretion (transport) of a mature DNase protein (e.g., SEQ ID NO: 2, 4, 6, 8, 10, 12 or variants thereof) and/or other proteins of interest. The signal sequence is typically located N-terminal to the precursor or mature protein sequence. For example, suitable signal sequences for use include, but are not limited to, signal sequences from secreted proteases, peptidases, amylases, glucoamylases, cellulases, lipases, esterases, arabinases, glucanases, chitosanases, lyases, xylanases, nucleases, phosphatases, transport and binding proteins, etc. In certain embodiments, a signal sequence is selected from an aprE signal sequence, a nprE signal sequence, a vpr signal sequence, a bglC signal sequence, a bglS signal sequence, a sacB signal sequence and amylase signal sequence, a heterologous signal sequence and/or a synthetic signal sequence,

Thus, in certain embodiments, standard techniques for transformation of microbial cells (which are well known to one skilled in the art) are used to transform a microbial host cell of the disclosure. Thus, the introduction of a DNA construct or vector into a host cell includes techniques such as transformation, electroporation, nuclear microinjection, transduction, transfection (e.g., lipofection mediated and DEAE-Dextrin mediated transfection), incubation with calcium phosphate DNA precipitate, high velocity bombardment with DNA-coated microprojectiles, gene gun or biolistic transformation, protoplast fusion and the like. General transformation techniques are known in the art (see, e.g., Ausubel et al., 1987, Sambrook et al., 2001 and 2012, and Campbell et al., 1989).

In certain embodiments, a heterologous gene, polynucleotide or ORF is cloned into an intermediate vector, before being transformed into a the microbial (host) cells for replication and/or expression. These intermediate vectors can be prokaryotic vectors, such as, e.g., plasmids, or shuttle vectors. Thus, the expression vector/construct typically contains a transcription unit or expression cassette that contains all the additional elements required for the expression of the heterologous sequence. For example, a typical expression cassette contains a 5′ promoter operably linked to the heterologous nucleic acid sequence encoding a protein of interest and may further comprise sequence signals required for efficient polyadenylation of the transcript, ribosome binding sites, and translation termination. Additional elements of the cassette may include enhancers and, if genomic DNA is used as the structural gene, introns with functional splice donor and acceptor sites.

The particular expression vector used to transport the genetic information into the cell is not particularly critical. Any of the conventional vectors used for expression in eukaryotic or prokaryotic cells may be used. Standard bacterial expression vectors include bacteriophages λ and M13, as well as plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. The elements that can be included in expression vectors may also be a replicon, a gene encoding antibiotic resistance to permit selection of bacteria that harbor recombinant plasmids, or unique restriction sites in nonessential regions of the plasmid to allow insertion of heterologous sequences.

The methods of transformation of the present invention may result in the stable integration of all or part of the transformation vector into the genome of the microbial cell. However, transformation resulting in the maintenance of a self-replicating extra-chromosomal transformation vector is also contemplated. Any of the known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other known methods for introducing cloned genomic DNA, cDNA, synthetic DNA or other foreign genetic material into a host cell (see, e.g., Sambrook et al., supra). Also of use is the Agrobacterium-mediated transfection method such as the one described in U.S. Pat. No. 6,255,115. It is only necessary that the particular genetic engineering procedure used be capable of successfully introducing at least one gene into the host cell capable of expressing the heterologous gene.

After the expression vector is introduced into the cells, the transfected cells are cultured under conditions favoring expression of the genes of interest. Large batches of transformed cells can be cultured as described herein. Finally, the broth and/or product(s) are recovered from the culture using standard techniques. Thus, the disclosure herein provides for the expression and secretion of desired proteins (e.g., DNase proteins, enzymes and the like).

Microbial cells of the disclosure may comprise genetic modifications of one or more endogenous genes and/or one or more introduced (heterologous) genes described herein. For example, microbial cells may be constructed to reduce or eliminate the expression of endogenous genes (e.g., reduce or eliminate genes encoding proteases), using methods well known in the art, e.g., insertions, disruptions, replacements, or deletions. The portion of the gene to be modified or inactivated may be, for example, the coding region or a regulatory element required for expression of the coding region.

In certain embodiments, a modified cell of the disclosure is constructed by introducing, substituting, or removing one or more nucleotides in the gene or a regulatory element required for the transcription or translation thereof. For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art.

In another embodiment, a modified cell is constructed by the process of gene conversion. For example, in the gene conversion method, a nucleic acid sequence corresponding to the gene(s) is mutagenized in vitro to produce a defective nucleic acid sequence, which is then transformed into the parental cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is effected by selection for the marker under conditions not permitting plasmid replication. Selection for a second recombination event leading to gene replacement is effected by examination of colonies for loss of the selectable marker and acquisition of the mutated gene. Alternatively, the defective nucleic acid sequence may contain an insertion, substitution, or deletion of one or more nucleotides of the gene, as described below.

In other embodiments, a modified cell is constructed by established anti-sense techniques using a nucleotide sequence complementary to the nucleic acid sequence of the gene. More specifically, expression of the gene by a cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to RNA interference (RNAi), small interfering RNA (siRNA), microRNA (miRNA), antisense oligonucleotides, and the like, all of which are well known to the skilled artisan.

In other embodiments, a modified cell is produced/constructed via CRISPR-Cas9 editing. For example, a gene of interest can be disrupted (or deleted or down-regulated) by means of nucleic acid guided endonucleases, that find their target DNA by binding either a guide RNA (e.g., Cas9) and Cpf1 or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete the gene. For example, the gene encoding the nucleic acid guided endonuclease (for this purpose Cas9 from S. pyogenes) or a codon optimized gene encoding the Cas9 nuclease is operably linked to a promoter active in the microbial cell and a terminator active in the microbial cell, thereby creating a microbial cell Cas9 expression cassette. Likewise, one or more target sites unique to the gene of interest are readily identified by a person skilled in the art. For example, to build a DNA construct encoding a gRNA-directed to a target site within the gene of interest, the variable targeting domain (VT) will comprise nucleotides of the target site which are 5′ of the (PAM) protospacer adjacent motif (TGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a microbial cell expression cassette for the gRNA is created by operably linking the DNA encoding the gRNA to a promoter active in the microbial cells and a terminator active in the microbial cells. The Cas9 expression cassette, the gRNA expression cassette and the editing template can be co-delivered to cells using many different methods (e.g., protoplast fusion, electroporation, natural competence, or induced competence). The transformed cells are screened by PCR amplifying the target gene locus, by amplifying the locus with a forward and reverse primer. These primers can amplify the wild-type locus or the modified locus that has been edited by the RGEN.

In yet other embodiments, a modified cell is constructed by random or specific mutagenesis using methods well known in the art, including, but not limited to, chemical mutagenesis and transposition. Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods. Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

### IV. Microbial Host Cells

As briefly stated above, certain embodiments are related to recombinant microbial (host) cells comprising and expressing heterologous polynucleotides which encode one or more DNase proteins of the disclosure. Certain other embodiments are directed to such microbial cells co-expressing (i) one or more DNase proteins and (ii) one or more proteins of interest. Microbial cells of the disclosure include Gram negative bacterial cells, Gram positive bacterial cells, filamentous fungal cells and yeast cell.

In certain embodiments, a Gram negative bacterial cell includes all bacteria of the classes of proteobacteria, such as alpha-proteobacteria, beta-proteobacteria, gamma-proteobacteria, delta-proteobacteria, epsilon-proteobacteria (e.g., including, but not limited to, proteobacteria such as Acidithiobacillales, Aeromonadales, Alteromonadales, Cardiobacteriales, Chromatiales, Enterbacteriales, Legionellales, Methylococcales, Oceanospirillales, Pasteurellales, Pseudomonadales, Thiotrichales, Vibrionales, Xanthomonadales. Enterobacteriales are Arsenophonus, Brenneria, Buchnera, Budvicia, Buttiauxella, Cedecea, Citrobacter, Dickeya, Edwardsiella, Enterobacter, Erwinia, Escherichia, Ewingella, Hafnia, Klebsiella, Kluyvera, Leclercia, Leminorella, Moellerella, Marganella, Obesumbacterium, Pantoea, Pectobacterium, Photorhabdus, Plesiomonas, Pragia, Proteus, Providencia, Rahnella, Raoultella, Saccharobacter, Salmonella, Samsonia, Serratia, Shigella, Sodalis, Tatumella, Thorsellia, Trabulsiella, Wigglesworthia, Xenorhabdus, Yersinia, Yokenella and the like).

In certain embodiments, a Gram positive bacterial cell includes the classes Bacilli, Clostridia and Mollicutes (e.g., including Lactobacillales with the families Aerococcaceae, Carnobacteriaceae, Enterococcaceae, Lactobacillaceae, Leuconostocaceae, Oscillospiraceae, Streptococcaceae and the Bacillales with the families Alicyclobacellaceae, Bacillaceae, Caryophanaceae, Listeriaceae, Paenibacillaceae, Planococcaceae, Sporolactobacillaceae, Staphylococcaceae, Thermoactinomycetaceae, Turicibacteraceae). In other embodiments, species of the family Bacillaceae include Alkalibacillus, Amphibacillus, Anoxybacillus, Bacillus, Caldalkalibacillus, Cerasilbacillus, Exiguobacterium, Filobacillus, Geobacillus, Gracilibacillus, Halobacillus, Halolactibacillus, Jeotgalibacillus, Lentibacillus, Marinibacillus, Oceanobacillus, Ornithinibacillus, Paraliobacillus, Paucisalibacillus, Pontibacillus, Pontibacillus, Saccharococcus, Salibacillus, Salinibacillus, Tenuibacillus, Thalassobacillus, Ureibacillus, Virgibacillus.

In other embodiments, Bacillus sp. cells include, but are not limited to, B. acidiceler, B. acidicola, B. acidocaldarius, B. acidoterrestris, B. aeolius, B. aerius, B. aerophilus, B. agaradhaerens. B. agri, B. aidingensis, B. akibai, B. alcalophilus, B. algicola, B. alginolyticus, B. alkalidiazo-trophicus, B. alkalinitrilicus, B. alkalitelluris, B. altitudinis, B. alveayuensis, B. alvei, B. amylolyticus, B. aneurinilyticus, B. aneurinolyticus, B. anthracia, B. aquimaris, B. arenosi, B. arseniciselenatis, B. arsenicoselenatis, B. arsenicus, B. arvi, B. asahii, B. atrophaeus, B. aurantiacus, B. axarquiensis, B. azotofixans, B. azotoformans, B. badius, B. barbaricus, B. bataviensis, B. beijingensis, B. benzoevorans, B. bogoriensis, B. boroniphilus, B. borstelenis, B. butanolivorans, B. carboniphilus, B. cecembensis, B. cellulosilyticus, B. centrosporus, B. chagannorensis, B. chitinolyticus, B. chondroitinus, B. choshinensis, B. cibi, B. circulans, B. clarkii, B. clausii, B. coagulans, B. coahuilensis, B. cohnii, B. curdianolyticus, B. cycloheptanicus, B. decisifrondis, B. decolorationis, B. dipsosauri, B. drentensis, B. edaphicus, B. ehimensis, B. endophyticus, B. farraginis, B. fastidiosus, B. firmus, B. plexus, B.foraminis, B. fordii, B. formosus, B. fortis, B. fumarioli, B. funiculus, B. fusiformis, B. galactophilus, B. galactosidilyticus, B. gelatini, B. gibsonii, B. ginsengi, B. ginsengihumi, B. globisporus, B. globisporus subsp. globisporus, B. globisporus subsp. marinus, B. glucanolyticus, B. gordonae, B. halmapalus, B. haloalkaliphilus, B. halodenitrificans, B. halodurans, B. halophilus, B. hemicellulosilyticus, B. herbersteinensis, B. horikoshii, B. horti, B. hemi, B. hwajinpoensis, B. idriensis, B. indicus, B. infantis, B. infernus, B. insolitus, B. isabeliae, B. jeotgali, B. kaustophilus, B. kobensis, B. koreensis, B. kribbensis, B krulwichiae, B. laevolacticus, B. larvae, B. laterosporus, B. lautus, B. lehensis, B. lentimorbus, B. lentus, B. litoralis, B. luciferensis, B. macauensis, B. macerans, B. macquariensis, B. macyae, B. malacitensis, B. mannanilyticus, B. marinus, B. marisflavi, B. marismortui, B. massiliensis, B. methanolicus, B. migulanus, B. mojavensis, B. mucilaginosus, B. muralis, B. murimartini, B. mycoides, B. naganoensis, B. nealsonii, B. neidei. B, niabensis, B. niacini, B. novalis, B. odysseyi, B. okhensis, B. okuhidensis, B. oleronius, B. oshimensis, B. pabuli, B. pallidus, B. pallidus (illeg.), B. panaciterrae, B. pantothenticus, B. parabrevis, B. pasteurii, B. patagoniensis, B. peoriae, B. plakortidis, B. pocheonensis, B. polygoni, B. polymyxa, B. popilliae, B. pseudalcaliphilus, B. pseudofirmus, B. pseudomycoides, B. psychrodurans, B. psychrophilus, B. psychrosaccarolyticus, B. psychrotolerans, B. pulvifaciens, B. pycnus, B. qingdaonensis, B. reuszeri, B. runs, B. safensis, B. salarius, B. salexigens, B. saliphilus, B. schlegelii, B. selenatarsenatis, B. selenitrireducens, B. seohaeanensis, B. shackletonii, B. silvestris, B. simplex, B. siralis, B. smithii, B. soli, B. sonorensis, B. sphaericus, B. sporothermodurans, B. stearothermophilus, B. stratosphericus, B. subterraneus, B. subtilis subsp. spizizenii, B. subtilis subsp. subtilis, B. taeanensis, B. tequilensis, B. thermantarcticus, B. thermoaerophilus, B. thermoamylovorans, B. thermoantarcticus, B. thermocatenulatus, B. thermocloacae, B. thermodenitrificans, B. thermoglucosidasius, B. thermoleovorans, B. thermoruber, B. thermosphaericus, B. thiaminolyticus, B. thioparans, B. thuringiensis, B. tusciae, B. validus, B. vallismortis, B. vedderi, B. velezensis, B. vietnamensis, B. vireti, B. vulcani, B. wakoensis and B. weihenstephanensis. In one preferred embodiment, the Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”.

Exemplary yeast (or yeast cells) includes budding yeast from the order Saccharomycetales.

Particular examples of yeast are Saccharomyces sp., including but not limited to S. cerevisiae. Filamentous fungal cells include, without limitation, Acremonium, Aspergillus, Emericella, Fusarium, Humicola, Mucor, Myceliophthora, Neurospora, Penicillium, Scytalidium, Thielavia, Tolypocladium, and Trichoderma species. In some embodiments, a filamentous fungus is a Trichoderma harzianum, Trichoderma koningii, Trichoderma longibrachiatum, Trichoderma reesei or Trichoderma viride. In other embodiments, a filamentous fungus may be an Aspergillus aculeatus, Aspergillus awamori, Aspergillus foetidus, Aspergillus japonicus, Aspergillus nidulans, Aspergillus niger, or Aspergillus oryzae.

### V. Fermenting Microbial Cells for the Production of Proteins

In certain embodiments, the present disclosure provides recombinant microbial cells capable of producing proteins of interest. More particularly, certain embodiments are related genetically modified microbial cells expressing heterologous polynucleotides encoding proteins having DNase activity, microbial cells co-expressing (i) proteins having DNase activity and (ii) one or more protein of interest, and the like. Thus, particular embodiments are related to cultivating (fermenting) microbial cells for the production of proteins having DNase activity and/or proteins of interest.

In general, fermentation methods well known in the art are used to ferment the microbial cells. In some embodiments, the cells are grown under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within batch cultures, cells progress through a static lag phase to a high growth log phase and finally to a stationary phase, where growth rate is diminished or halted. If untreated, cells in the stationary phase eventually die. In general, cells in log phase are responsible for the bulk of production of product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and well known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Culturing/fermenting is generally accomplished in a growth medium comprising an aqueous mineral salts medium, organic growth factors, a carbon and energy source material, molecular oxygen, and, of course, a starting inoculum of the microbial host to be employed.

In addition to the carbon and energy source, oxygen, assimilable nitrogen, and an inoculum of the microorganism, it is necessary to supply suitable amounts in proper proportions of mineral nutrients to assure proper microorganism growth, maximize the assimilation of the carbon and energy source by the cells in the microbial conversion process, and achieve maximum cellular yields with maximum cell density in the fermentation media.

The composition of the aqueous mineral medium can vary over a wide range, depending in part on the microorganism and substrate employed, as is known in the art. The mineral media should include, in addition to nitrogen, suitable amounts of phosphorus, magnesium, calcium, potassium, sulfur, and sodium, in suitable soluble assimilable ionic and combined forms, and also present preferably should be certain trace elements such as copper, manganese, molybdenum, zinc, iron, boron, and iodine, and others, again in suitable soluble assimilable form, all as known in the art.

The fermentation reaction is an aerobic process in which the molecular oxygen needed is supplied by a molecular oxygen-containing gas such as air, oxygen-enriched air, or even substantially pure molecular oxygen, provided to maintain the contents of the fermentation vessel with a suitable oxygen partial pressure effective in assisting the microorganism species to grow in a thriving fashion.

The fermentation temperature can vary somewhat, but for most microbial cells the temperature generally will be within the range of about 20° C. to 40° C.

The microorganisms also require a source of assimilable nitrogen. The source of assimilable nitrogen can be any nitrogen-containing compound or compounds capable of releasing nitrogen in a form suitable for metabolic utilization by the microorganism. While a variety of organic nitrogen source compounds, such as protein hydrolysates, can be employed, usually cheap nitrogen-containing compounds such as ammonia, ammonium hydroxide, urea, and various ammonium salts such as ammonium phosphate, ammonium sulfate, ammonium pyrophosphate, ammonium chloride, or various other ammonium compounds can be utilized. Ammonia gas itself is convenient for large scale operations, and can be employed by bubbling through the aqueous ferment (fermentation medium) in suitable amounts. At the same time, such ammonia can also be employed to assist in pH control.

The pH range in the aqueous microbial ferment (fermentation admixture) should be in the exemplary range of about 2.0 to 8.0. Preferences for pH range of microorganisms are dependent on the media employed to some extent, as well as the particular microorganism, and thus change somewhat with change in media as can be readily determined by those skilled in the art.

Preferably, the fermentation is conducted in such a manner that the carbon-containing substrate can be controlled as a limiting factor, thereby providing good conversion of the carbon-containing substrate to cells and avoiding contamination of the cells with a substantial amount of unconverted substrate. The latter is not a problem with water-soluble substrates, since any remaining traces are readily washed off. It may be a problem, however, in the case of non-water-soluble substrates, and require added product-treatment steps such as suitable washing steps.

As described above, the time to reach this level is not critical and may vary with the particular microorganism and fermentation process being conducted. However, it is well known in the art how to determine the carbon source concentration in the fermentation medium and whether or not the desired level of carbon source has been achieved.

If desired, part or all of the carbon and energy source material and/or part of the assimilable nitrogen source such as ammonia can be added to the aqueous mineral medium prior to feeding the aqueous mineral medium to the fermenter.

Each of the streams introduced into the reactor preferably is controlled at a predetermined rate, or in response to a need determinable by monitoring such as concentration of the carbon and energy substrate, pH, dissolved oxygen, oxygen or carbon dioxide in the off-gases from the fermenter, cell density measurable by dry cell weights, light transmittancy, or the like. The feed rates of the various materials can be varied so as to obtain as rapid a cell growth rate as possible, consistent with efficient utilization of the carbon and energy source, to obtain as high a yield of microorganism cells relative to substrate charge as possible.

In either a batch, or the preferred fed batch operation, all equipment, reactor, or fermentation means, vessel or container, piping, attendant circulating or cooling devices, and the like, are initially sterilized, usually by employing steam such as at about 121° C. for at least about 15 minutes. The sterilized reactor then is inoculated with a culture of the selected microorganism in the presence of all the required nutrients, including oxygen, and the carbon-containing substrate. The type of fermenter employed is not critical.

### VI. Broth Conditioning, Protein Recovery Processes and Uses Thereof

As generally described above, certain aspects of the disclosure are related to cultivating (fermenting) microbial cells for the production of proteins having DNase activity and/or proteins of interest.

Certain embodiments are therefore related to fermentation broths obtained by fermenting microbial cells expressing one or more proteins having DNase activity. Certain other embodiments are related to fermentation broths obtained by fermenting microbial cells co-expressing (i) one or more proteins of interest and (ii) one or more proteins having DNase activity (e.g., comprising at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO: 16 or SEQ ID NO: 17).

More particularly, as set forth in the Examples herewith, such microbial cell fermentation broths comprising DNase activity are particularly useful in degrading contaminating DNA according to many aspects of the disclosure. In related embodiments, such microbial cell fermentation broths are further subjected to a protein recovery process. Certain other aspects are therefore directed to compositions and methods for producing a protein of interest (POI) essentially free from contaminating DNA. For example, certain embodiments provide protein preparations essentially free from contaminating DNA produced according to the compositions and methods of the disclosure.

Thus, in other embodiments, the disclosure relates to methods for recovering proteins of interest from microbial cell fermentation broths, such as (a) obtaining a microbial cell fermentation broth comprising a POI, (b) treating the broth with an exogenously introduced protein preparation comprising one or more proteins having DNase activity, and (c) recovering the POI from the broth, wherein the recovered protein(s) is/are essentially free from contaminating DNA.

Certain other embodiments therefore provide protein preparations (or proteins isolated therefrom) essentially free from contaminating DNA, recovered according to the disclosed methods and compositions herein.

Thus, certain other embodiments relate to methods for reducing the DNA content of a fermentation broth in which microbial host cells have been fermented comprising introducing into the fermentation broth an exogenous protein preparation comprising one or more proteins of SEQ ID NO: 2, SEQ ID NO: 4, SEQ ID NO: 6, SEQ ID NO: 8, SEQ ID NO: 10, SEQ ID NO: 12, SEQ ID NO: 16 or SEQ ID NO: 17 (or active variants thereof). In other embodiments, one or more (multiple) microbial cell fermentation broths (e.g., comprising one or more proteins of interest) may be combined (mixed) in the presence of exogenous DNase protein preparations. In related embodiments, the combined fermentation broth(s) is/are further subjected to one or more protein recovery steps performed in the presence of at least one exogenously introduced protein preparations comprising one or more proteins having at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO: 16 or SEQ ID NO: 17.

More particularly, as described herein, one or more proteins having DNase activity, and/or one or more proteins of interest can be secreted into the fermentation broth and/or retained intracellularly. Therefore, in certain aspects, the fermentation broth is subjected to at least one the protein recovery process (step) selected from a cell lysis process, a cell separation process, a protein concentration process and/or a protein purification process. More particularly, in certain aspects, the fermentation broth is subjected to at least one the protein recovery process performed in the presence of an exogenously introduced DNase protein preparation. The recovery of proteins from a fermentation broth can be done by procedures known to one of skill in the art to obtain a desired protein preparation.

The fermentation broth will generally contain cellular debris, including cells, various suspended solids and other biomass contaminants, as well as the desired protein (e.g., enzyme) products and/or DNase proteins. For example, suitable protein recovery processes include, but are not limited to, conventional solid-liquid separation techniques such as, e.g., centrifugation, filtration, dialysis, microfiltration, rotary vacuum filtration, or other known processes, to produce a cell-free filtrate.

The terms “cell separation” or “cell separation process” are not meant to be limiting, and include methods of cell separation and broth clarification know to those skilled in the art, such as centrifugation, rotary vacuum drum filtration, filter pressing, microfiltration and the like. Likewise, the terms “concentration” or “concentration process” are not meant to be limiting, and include concentration methods know to those skilled in the art, such as ultrafiltration, evaporation, centrifugation and the like. In certain aspects, a microbial cell fermentation broth comprising a protein of interest is treated for a sufficient amount of time with an introduced (exogenous) DNase protein preparation, wherein the protein of interest is recovered from the broth following such DNase treatment, and the protein of interest recovered therefrom is essentially free from contaminating DNA. For example, in certain embodiments, the broth is treated with a DNase protein preparation for a sufficient amount of time to render the broth essentially free from contaminating DNA. One skilled in the art may monitor DNA content of the broth using known techniques and adjust the amount of time accordingly and/or the total activity of a particular DNase preparation. In certain embodiments, a sufficient amount of time is about one (1) second to about forty-eight (48) hours.

It may be preferable to further concentrate the fermentation broth or the cell-free filtrate prior to crystallization using techniques such as ultrafiltration, evaporation or precipitation. Precipitating the proteinaceous components of the supernatant or filtrate may be accomplished by means of a salt, e.g., ammonium sulfate, followed by purification by a variety of chromatographic procedures, e.g., ion exchange chromatography, affinity chromatography or similar art recognized procedures.

Thus, as generally set forth above, protein preparations according to the instant disclosure may be recovered, purified, enriched and the like using methods known to one skilled the art (e.g., art-recognized separation techniques such as ion exchange chromatography, affinity chromatography, hydrophobic separation, dialysis, protease treatment, ammonium sulphate precipitation (or other protein salt precipitation), centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

With regard to Gram negative bacterial cells (e.g., E. coli), purification steps for separating endotoxins (lipopolysaccharides; LPS) which are known in the art may can be used (e.g., anion exchange chromatography; affinity chromatography; ion exchange chromatography, in particular ion exchange chromatography using alkanediol; ultrafiltration; purification using affinity adsorbents such as, e.g., L-histidine, poly-L-histidine, poly(gamma-methyl L-glutamate), polymyxin B; gel filtration; gel filtration chromatography; sucrose gradient centrifugation; purification using dual-phase micelle systems; triton X-114-based phase separation; temperature-induced phase separation; purification by a non-selective adsorption with hydrophobic adsorbents or anion exchangers; polyacrylamide gel electrophoresis, in particular slab polyacrylamide gel electrophoresis; SDS gel electrophoresis; membrane-based chromatography; agarose gel electrophoresis; caesium chloride gradient centrifugation; affinity purification using beads).

Thus, in certain embodiments, a protein preparation comprises at least one (one or more) DNase protein of the disclosure (e.g., SEQ ID NO: 2, SEQ ID NO: 4, SEQ ID NO: 6, SEQ ID NO: 8, SEQ ID NO: 10, SEQ ID NO: 12, SEQ ID NO: 16 or SEQ ID NO: 17, or active DNA variants thereof, or active fragments thereof). In certain other embodiments, a protein preparation comprises at least one (one or more) DNase protein and a protein of interest (e.g., an enzyme) or multiple proteins of interest (e.g., enzyme combinations/blends). Thus, in other embodiments, a protein preparation obtained from a microbial cell fermentation broth comprises a protein of interest (e.g., an enzyme) or multiple proteins of interest (e.g., enzyme combinations/blends). The protein preparations of the disclosure (e.g., a DNase preparation, an enzyme (POI) preparation, a combined DNase/enzyme (POI) preparation, etc.) can be solid (e.g., a lyophilized powder), paste-like or liquid (e.g., an aqueous solution or dispersion).

Therefore, certain aspects of the disclosure provide novel DNase compositions suitable for degrading contaminating DNA. In certain embodiments, novel DNases are co-expressed with a protein of interest (POI), wherein the expressed DNases degrade contaminating DNA present in the broth. In other aspects, novel DNases are expressed in a microbial cell of the disclosure, wherein DNase (protein) preparations are obtained, derived or recovered from the broth. In certain aspects, a DNase preparation is a liquid preparation, such as concentrated broth (e.g., a DNase UFC). In related embodiments, a liquid DNase preparation is used to treat a protein preparation comprising a protein of interest. For example, in certain aspects, liquid DNase preparations are used to treat a protein of interest (i.e., a protein preparation comprising the POI) during a POI purification process and/or a POI formulation process, thereby removing contaminating DNA from the purified and/or formulated POI. As further demonstrated in the Examples below, the DNases of the disclosure are particularly useful for degrading contaminating DNA present in microbial cell fermentation broths. In certain aspects, a DNase preparation is added to a microbial cell fermentation broth comprising a protein of interest (POI). In certain embodiments, the broth comprising the POI is collected. In related aspects, the collected broth is harvested and/or the collected broth is treated. In any of these aspects or embodiments of the disclosure, a DNase preparation may be added to remove/degrade contaminating DNA therein. For example, broth treatments include, but are not limited to, broth stabilization processes, broth pH optimization, broth temperature optimization, broth additives, broth holding times, and the like.

### VI. Proteins of Interest

A protein of interest (POI) of the instant disclosure can be any endogenous or heterologous protein, and it may be a variant of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest. Thus, in certain embodiments, a modified cell of the disclosure expresses an endogenous POI, a heterologous POI, or a combination of one or more of such POIs.

In certain embodiments, a modified cell may produce an increased amount of a POI (e.g., protein having DNase activity) relative to a parental (control) cell, wherein the increased amount of the POI is at least about a 0.01% increase, at least about a 0.10% increase, at least about a 0.50% increase, at least about a 1.0% increase, at least about a 2.0% increase, at least about a 3.0% increase, at least about a 4.0% increase, at least about a 5.0% increase, or an increase greater than 5.0%. In certain embodiments, the increased amount of the POI is determined by assaying enzymatic activity and/or by assaying/quantifying the specific productivity (Qp) thereof. Likewise, one skilled in the art may utilize other routine methods and techniques known in the art for detecting, assaying, measuring, etc. the expression, production or secretion of one or more proteins of interest.

In certain embodiments, a POI or a variant POI thereof is selected from the group consisting of acetyl esterases, aminopeptidases, amylases, arabinases, arabinofuranosidases, arylesterases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, lysozymes, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phosphodiesterases, phytases, polyesterases, polygalacturonases, proteases, peptidases, rhamno-galacturonases, ribonucleases, transferases, transport proteins, transglutaminases, xylanases, hexose oxidases, and combinations thereof.

Thus, in certain embodiments, a POI or a variant POI thereof is an enzyme selected from Enzyme Commission (EC) Number EC 1, EC 2, EC 3, EC 4, EC 5 or EC 6.

For example, in certain embodiments a POI is an oxidoreductase enzyme, including, but not limited to, an EC 1 (oxidoreductase) enzyme selected from EC 1.10.3.2 (e.g., a laccase), EC 1.10.3.3 (e.g., L-ascorbate oxidase), EC 1.1.1.1 (e.g., alcohol dehydrogenase), EC 1.11.1.10 (e.g., chloride peroxidase), EC 1.11.1.17 (e.g., peroxidase), EC 1.1.1.27 (e.g., L-lactate dehydrogenase), EC 1.1.1.47 (e.g., glucose 1-dehydrogenase), EC 1.1.3.X (e.g., glucose oxidase), EC 1.1.3.10 (e.g., pyranose oxidase), EC 1.13.11.X (e.g., dioxygenase), EC 1.13.11.12 (e.g., lineolate 13S-lipozygenase), EC 1.1.3.13 (e.g., alcohol oxidase), EC 1.14.14.1 (e.g., monooxygenase), EC 1.14.18.1 (e.g., monophenol monooxigenase) EC 1.15.1.1 (e.g., superoxide dismutase), EC 1.1.5.9 (formerly EC 1.1.99.10, e.g., glucose dehydrogenase), EC 1.1.99.18 (e.g., cellobiose dehydrogenase), EC 1.1.99.29 (e.g., pyranose dehydrogenase), EC 1.2.1.X (e.g., fatty acid reductase), EC 1.2.1.10 (e.g., acetaldehyde dehydrogenase), EC 1.5.3.X (e.g., fructosyl amine reductase), EC 1.8.1.X (e.g., disulfide reductase) and EC 1.8.3.2 (e.g., thiol oxidase).

In certain embodiments a POI is a transferase enzyme, including, but not limited to, an EC 2 (transferase) enzyme selected from EC 2.3.2.13 (e.g., transglutaminase), EC 2.4.1.X (e.g., hexosyltransferase), EC 2.4.1.40 (e.g., alternasucrase), EC 2.4.1.18 (e.g., 1,4 alpha-glucan branching enzyme), EC 2.4.1.19 (e.g., cyclomaltodextrin glucanotransferase), EC 2.4.1.2 (e.g., dextrin dextranase), EC 2.4.1.20 (e.g., cellobiose phosphorylase), EC 2.4.1.25 (e.g., 4-alpha-glucanotransferase), EC 2.4.1.333 (e.g., 1,2-beta-oligoglucan phosphor transferase), EC 2.4.1.4 (e.g., amylosucrase), EC 2.4.1.5 (e.g., dextransucrase), EC 2.4.1.69 (e.g., galactoside 2-alpha-L-fucosyl transferase), EC 2.4.1.9 (e.g., inulosucrase), EC 2.7.1.17 (e.g., xylulokinase), EC 2.7.7.89 (formerly EC 3.1.4.15, e.g., [glutamine synthetase]-adenylyl-L-tyrosine phosphorylase), EC 2.7.9.4 (e.g., alpha glucan kinase) and EC 2.7.9.5 (e.g., phosphoglucan kinase).

In other embodiments a POI is a hydrolase enzyme, including, but not limited to, an EC 3 (hydrolase) enzyme selected from EC 3.1.X.X (e.g., an esterase), EC 3.1.1.1 (e.g., pectinase), EC 3.1.1.14 (e.g., chlorophyllase), EC 3.1.1.20 (e.g., tannase), EC 3.1.1.23 (e.g., glycerol-ester acylhydrolase), EC 3.1.1.26 (e.g., galactolipase), EC 3.1.1.32 (e.g., phospholipase A1), EC 3.1.1.4 (e.g., phospholipase A2), EC 3.1.1.6 (e.g., acetylesterase), EC 3.1.1.72 (e.g., acetylxylan esterase), EC 3.1.1.73 (e.g., feruloyl esterase), EC 3.1.1.74 (e.g., cutinase), EC 3.1.1.86 (e.g., rhamnogalacturonan acetylesterase), EC 3.1.1.87 (e.g., fumosin B1 esterase), EC 3.1.26.5 (e.g., ribonuclease P), EC 3.1.3.X (e.g., phosphoric monoester hydrolase), EC 3.1.30.1 (e.g., Aspergillus nuclease Si), EC 3.1.30.2 (e.g., Serratia marcescens nuclease), EC 3.1.3.1 (e.g., alkaline phosphatase), EC 3.1.3.2 (e.g., acid phosphatase), EC 3.1.3.8 (e.g., 3-phytase), EC 3.1.4.1 (e.g., phosphodiesterase I), EC 3.1.4.11 (e.g., phosphoinositide phospholipase C), EC 3.1.4.3 (e.g., phospholipase C), EC 3.1.4.4 (e.g., phospholipase D), EC 3.1.6.1 (e.g., arylsufatase), EC 3.1.8.2 (e.g., diisopropyl-fluorophosphatase), EC 3.2.1.10 (e.g., oligo-1,6-glucosidase), EC 3.2.1.101 (e.g., mannan endo-1,6-alpha-mannosidase), EC 3.2.1.11 (e.g., alpha-1,6-glucan-6-glucanohydrolase), EC 3.2.1.131 (e.g., xylan alpha-1,2-glucuronosidase), EC 3.2.1.132 (e.g., chitosan N-acetylglucosaminohydrolase), EC 3.2.1.139 (e.g., alpha-glucuronidase), EC 3.2.1.14 (e.g., chitinase), EC 3.2.1.151 (e.g., xyloglucan-specific endo-beta-1,4-glucanase), EC 3.2.1.155 (e.g., xyloglucan-specific exo-beta-1,4-glucanase), EC 3.2.1.164 (e.g., galactan endo-1,6-beta-galactosidase), EC 3.2.1.17 (e.g., lysozyme), EC 3.2.1.171 (e.g., rhamnogalacturonan hydrolase), EC 3.2.1.174 (e.g., rhamnogalacturonan rhamnohydrolase), EC 3.2.1.2 (e.g., beta-amylase), EC 3.2.1.20 (e.g., alpha-glucosidase), EC 3.2.1.22 (e.g., alpha-galactosidase), EC 3.2.1.25 (e.g., beta-mannosidase), EC 3.2.1.26 (e.g., beta-fructofuranosidase), EC 3.2.1.37 (e.g., xylan 1,4-beta-xylosidase), EC 3.2.1.39 (e.g., glucan endo-1,3-beta-D-glucosidase), EC 3.2.1.40 (e.g., alpha-L-rhamnosidase), EC 3.2.1.51 (e.g., alpha-L-fucosidase), EC 3.2.1.52 (e.g., beta-N-Acetylhexosaminidase), EC 3.2.1.55 (e.g., alpha-N-arabinofuranosidase), EC 3.2.1.58 (e.g., glucan 1,3-beta-glucosidase), EC 3.2.1.59 (e.g., glucan endo-1,3-alpha-glucosidase), EC 3.2.1.67 (e.g., galacturan 1,4-alpha-galacturonidase), EC 3.2.1.68 (e.g., isoamylase), EC 3.2.1.7 (e.g., 1-beta-D-fructan fructanohydrolase), EC 3.2.1.74 (e.g., glucan 1,4-β-glucosidase), EC 3.2.1.75 (e.g., glucan endo-1,6-beta-glucosidase), EC 3.2.1.77 (e.g., mannan 1,2-(1,3)-alpha-mannosidase), EC 3.2.1.80 (e.g., fructan beta-fructosidase), EC 3.2.1.82 (e.g., exo-poly-alpha-galacturonosidase), EC 3.2.1.83 (e.g., kappa-carrageenase), EC 3.2.1.89 (e.g., arabinogalactan endo-1,4-beta-galactosidase), EC 3.2.1.91 (e.g., cellulose 1,4-beta-cellobiosidase), EC 3.2.1.96 (e.g., mannosyl-glycoprotein endo-beta-N-acetylglucosaminidase), EC 3.2.1.99 (e.g., arabinan endo-1,5-alpha-L-arabinanase), EC 3.4.X.X (e.g., peptidase), EC 3.4.11.X (e.g., aminopeptidase), EC 3.4.11.1 (e.g., leucyl aminopeptidase), EC 3.4.11.18 (e.g., methionyl aminopeptidase), EC 3.4.13.9 (e.g., Xaa-Pro dipeptidase), EC 3.4.14.5 (e.g., dipeptidyl-peptidase IV), EC 3.4.16.X (e.g., serine-type carboxypeptidase), EC 3.4.16.5 (e.g., carboxypeptidase C), EC 3.4.19.3 (e.g., pyroglutamyl-peptidase I), EC 3.4.21.X (e.g., serine endopeptidase), EC 3.4.21.1 (e.g., chymotrypsin), EC 3.4.21.19 (e.g., glutamyl endopeptidase), EC 3.4.21.26 (e.g., prolyl oligopeptidase), EC 3.4.21.4 (e.g., trypsin), EC 3.4.21.5 (e.g., thrombin), EC 3.4.21.63 (e.g., oryzin), EC 3.4.21.65 (e.g., thermomycolin), EC 3.4.21.80 (e.g., streptogrisin A), EC 3.4.22.X (e.g., cysteine endopeptidase), EC 3.4.22.14 (e.g., actinidain), EC 3.4.22.2 (e.g., papain), EC 3.4.22.3 (e.g., ficain), EC 3.4.22.32 (e.g., stem bromelain), EC 3.4.22.33 (e.g., fruit bromelain), EC 3.4.22.6 (e.g., chymopapain), EC 3.4.23.1 (e.g., pepsin A), EC 3.4.23.2 (e.g., pepsin B), EC 3.4.23.22 (e.g., endothiapepsin), EC 3.4.23.23 (e.g., mucorpepsin), EC 3.4.23.3 (e.g., gastricsin), EC 3.4.24.X (e.g., metalloendopeptidase), EC 3.4.24.39 (e.g., deuterolysin), EC 3.4.24.40 (e.g., serralysin), EC 3.5.1.1 (e.g., asparaginase), EC 3.5.1.11 (e.g., penicillin amidase), EC 3.5.1.14 (e.g., N-acyl-aliphatic-L-amino acid amidohydrolase), EC 3.5.1.2 (e.g., L-glutamine amidohydrolase), EC 3.5.1.28 (e.g., N-acetylmuramoyl-L-alanine amidase), EC 3.5.1.4 (e.g., amidase), EC 3.5.1.44 (e.g., protein-L-glutamine amidohydrolase), EC 3.5.1.5 (e.g., urease), EC 3.5.1.52 (e.g., peptide-N(4)-(N-acetyl-beta-glucosaminyl)asparagine amidase), EC 3.5.1.81 (e.g., N-Acyl-D-amino-acid deacylase), EC 3.5.4.6 (e.g., AMP deaminase) and EC 3.5.5.1 (e.g., nitrilase).

In other embodiments a POI is a lyase enzyme, including, but not limited to, an EC 4 (lyase) enzyme selected from EC 4.1.2.10 (e.g., mandelonitrile lyase), EC 4.1.3.3 (e.g., N-acetylneuraminate lyase), EC 4.2.1.1 (e.g., carbonate dehydratase), EC 4.2.2.—(e.g., rhamnogalacturonan lyase), EC 4.2.2.10 (e.g., pectin lyase), EC 4.2.2.22 (e.g., pectate trisaccharide-lyase), EC 4.2.2.23 (e.g., rhamnogalacturonan endolyase) and EC 4.2.2.3 (e.g., mannuronate-specific alginate lyase).

In certain other embodiments a POI is an isomerase enzyme, including, but not limited to, an EC 5 (isomerase) enzyme selected from EC 5.1.3.3 (e.g., aldose 1-epimerase), EC 5.1.3.30 (e.g., D-psicose 3-epimerase), EC 5.4.99.11 (e.g., isomaltulose synthase) and EC 5.4.99.15 (e.g., (1→4)-α-D-glucan 1-α-D-glucosylmutase).

In yet other embodiments, a POI is a ligase enzyme, including, but not limited to, an EC 6 (ligase) enzyme selected from EC 6.2.1.12 (e.g., 4-coumarate:coenzyme A ligase) and EC 6.3.2.28 (e.g., L-amino-acid alpha-ligase)9

Thus, in certain embodiments, industrial protease producing Bacillus host cells provide particularly preferred expression hosts. Likewise, in certain other embodiments, industrial amylase producing Bacillus host cells provide particularly preferred expression hosts.

For example, there are two general types of proteases which are typically secreted by Bacillus spp., namely neutral (or “metalloproteases”) and alkaline (or “serine”) proteases. For example, Bacillus subtilisin proteins (enzymes) are exemplary serine proteases for use in the present disclosure. A wide variety of Bacillus subtilisins have been identified and sequenced, for example, subtilisin 168, subtilisin BPN′, subtilisin Carlsberg, subtilisin DY, subtilisin 147 and subtilisin 309 (e.g., WO 1989/06279). In some embodiments of the present disclosure, the modified Bacillus cells produce mutant (i.e., variant) proteases. Numerous references provide examples of variant proteases, such as PCT Publication Nos. WO1999/20770; WO1999/20726; WO1999/20769; WO1989/06279; U.S. RE34,606; U.S. Pat. Nos. 4,914,031; 4,980,288; 5,208,158; 5,310,675; 5,336,611; 5,399,283; 5,441,882; 5,482,849; 5,631,217; 5,665,587; 5,700,676; 5,741,694; 5,858,757; 5,880,080; 6,197,567 and 6,218,165. Thus, in certain embodiments, a modified Bacillus cells of the disclosure comprises an expression construct encoding a protease.

In certain other embodiments, a modified Bacillus cells of the disclosure comprises an expression construct encoding an amylase. A wide variety of amylase enzymes and variants thereof are known to one skilled in the art. For example, International PCT Publication NO. WO2006/037484 and WO 2006/037483 describe variant α-amylases having improved solvent stability, Publication No. WO1994/18314 discloses oxidatively stable α-amylase variants, Publication No. WO1999/19467, WO2000/29560 and WO2000/60059 disclose Termamyl-like α-amylase variants, Publication No. WO2008/112459 discloses α-amylase variants derived from Bacillus sp. number 707, Publication No. WO1999/43794 discloses maltogenic α-amylase variants, Publication No. WO1990/11352 discloses hyper-thermostable α-amylase variants, Publication No. WO2006/089107 discloses α-amylase variants having granular starch hydrolyzing activity.

In other embodiments, a POI or variant POI expressed and produced in a modified cell of the disclosure is a peptide, a peptide hormone, a growth factor, a clotting factor, a chemokine, a cytokine, a lymphokine, an antibody, a receptor, an adhesion molecule, a microbial antigen (e.g., HBV surface antigen, HPV E7, etc.), variants thereof, fragments thereof and the like. Other types of proteins (or variants thereof) of interest may be those that are capable of providing nutritional value to a food or to a crop. Non-limiting examples include plant proteins that can inhibit the formation of anti-nutritive factors and plant proteins that have a more desirable amino acid composition (e.g., a higher lysine content than a non-transgenic plant).

There are various assays known to those of ordinary skill in the art for detecting and measuring activity of intracellularly and extracellularly expressed proteins. In particular, for proteases, there are assays based on the release of acid-soluble peptides from casein or hemoglobin measured as absorbance at 280 nm or colorimetrically, using the Folin method. Other exemplary assays include succinyl-Ala-Ala-Pro-Phe-para-nitroanilide assay (SAAPFpNA) and the 2,4,6-trinitrobenzene sulfonate sodium salt assay (TNBS assay).

International PCT Publication No. WO2014/164777 discloses Ceralpha α-amylase activity assays useful for amylase activities described herein.

Means for determining the levels of secretion of a protein of interest in a host cell and detecting expressed proteins include the use of immunoassays with either polyclonal or monoclonal antibodies specific for the protein. Examples include enzyme-linked immunosorbent assay (ELISA), radioimmunoassay (RIA), fluorescence immunoassay (FIA), and fluorescent activated cell sorting (FACS).

### VII. Exemplary Embodiments

Non-limiting embodiments of compositions and methods disclosed herein are as follows:

1. An isolated nucleic acid comprising at least 80% identity to a nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO:14 or SEQ ID NO:15.

2. An isolated nucleic acid comprising at least 80% identity to a nucleotide sequence of SEQ ID NO: 1, SEQ ID NO: 3, SEQ ID NO: 5, SEQ ID NO: 7, SEQ ID NO: 9 or SEQ ID NO: 11, and encoding a protein comprising at least 85% identity to a protein of SEQ ID NO: 2, SEQ ID NO: 4, SEQ ID NO: 6, SEQ ID NO: 8, SEQ ID NO: 10 or SEQ ID NO: 12, respectively.

3. The nucleic acid of embodiment 2, wherein the encoded protein comprises deoxyribonuclease (DNase) activity.

4. A vector comprising the nucleic acid of any one of embodiments 1-3.

5. A recombinant microbial cell comprising the vector of embodiment 4.

6. An isolated protein comprising at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17.

7. The protein of embodiment 6, comprising deoxyribonuclease (DNase) activity.

8. The protein of embodiment 6, comprising a HNH nuclease superfamily domain.

9. The protein of embodiment 6, further defined as protease resistant.

10. A protein preparation comprising one or more proteins of embodiment 6.

11. A polynucleotide comprising an upstream (5′) promoter sequence operably linked to a downstream (3′) nucleic acid encoding a protein comprising at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17.

12. A polynucleotide comprising an upstream (5′) promoter sequence operably linked to a downstream (3′) nucleic acid encoding a protein signal sequence operably linked to a downstream (3′) nucleic acid encoding a protein comprising at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17.

13. The polynucleotide of embodiment 11 or embodiment 12, further comprising a terminator sequence positioned downstream (3′) and operably linked to the nucleic acid encoding the protein.

14. A recombinant microbial cell expressing one or more proteins comprising at least 85% identity to a protein sequence of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17.

15. A recombinant microbial cell co-expressing a protein of interest (POI) and one or more proteins comprising at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17.

16. The recombinant cell of embodiment 15, wherein the POI is selected from the group consisting of a lyase, a ligase, a lectin, a hydrolase, an oxidoreductase, a transferase, an isomerase, an antibody, a receptor, and a cytokine.

17. The recombinant cell of embodiment 15, wherein the POI is an animal feed protein or a food enzyme.

18. The recombinant cell of embodiment 17, wherein the animal feed protein is selected from the group consisting of phytases, proteases, cellulases, β-glucanases, xylanases, lipases, mannanases, α-galactosidases, pectinases, and amylases.

19. The recombinant cell of embodiment 17, wherein the food enzyme is selected from the group consisting of lactases, amylases, proteases, cellulases, lipases and xylanases.

20. The recombinant cell of embodiment 14 or embodiment 15, selected from a Gram-negative bacterial cell, a Gram-positive bacterial cell, a filamentous fungal cell, or a yeast cell.

21. The recombinant cell of embodiment 20, wherein the Gram-negative cell is an E. coli cell.

22. The recombinant cell of embodiment 20, wherein the Gram-positive bacterial cell is a Bacillus sp. cell.

23. The recombinant cell of embodiment 20, wherein the filamentous fungal cell is selected from an Aspergillus sp. cell, a Myceliophthora sp. cell and a Trichoderma sp. cell.

24. The recombinant cell of embodiment 20, wherein the yeast cell is selected from a Yarrowia sp. cell or a Saccharomyces sp. cell.

25. A fermentation broth obtained by fermenting a microbial cell expressing one or more proteins comprising at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17.

26. The broth of embodiment 25, for use in degrading contaminating DNA present in a protein preparation.

27. The broth of embodiment 25, wherein broth is subjected to a broth conditioning process.

28. The conditioned broth of embodiment 27, for use in degrading contaminating DNA present in a protein preparation.

29. The broth of embodiment 25 or embodiment 27, wherein broth is subjected to a protein recovery process.

30. The recovered broth of embodiment 29, for use in degrading contaminating DNA present in a protein preparation.

31. A fermentation broth obtained by fermenting a microbial cell co-expressing (i) one or more proteins of interest and (ii) one or more proteins comprising at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17.

32. The broth of embodiment 31, wherein the one or more proteins of interest isolated from the broth are essentially free of contaminating DNA.

33. The broth of embodiment 31, wherein broth is subjected to a broth conditioning process, wherein the one or more proteins of interest isolated from the conditioned broth are essentially free of contaminating DNA.

34. The broth of embodiment 33, wherein the conditioned broth is subjected to a protein recovery process.

35. The broth of embodiment 34, wherein the one or more proteins of interest isolated from the recovered broth are essentially free of contaminating DNA.

36. A method for producing a protein of interest (POI) essentially free from contaminating DNA comprising (a) obtaining or constructing a microbial cell expressing a POI and modifying the cell to express one or more DNase proteins comprising at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17, (b) fermenting the modified cell under suitable conditions for the expression of the POI and the one or more DNase proteins, and (c) harvesting the fermentation broth at the end of fermentation, wherein the POI isolated from the harvested broth is essentially free from contaminating DNA.

37. The method of embodiment 36, wherein the harvested broth is subjected to a broth conditioning process and/or a protein recovery process.

38. The method of embodiment 36, wherein the one or more expressed DNase proteins are protease resistant.

39. A protein of interest preparation essentially free from contaminating DNA produced according to the method of embodiment 36.

40. A method for producing a protein of interest (POI) essentially free from contaminating DNA comprising (a) obtaining or constructing a microbial cell expressing a POI and fermenting the cell under suitable conditions for the expression of the POI, and (b) harvesting the fermentation broth at the end of fermentation, wherein the harvested broth is treated with a DNase preparation comprising one or more DNase proteins having at least 85% identity to SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17, wherein the DNase treated broth is essentially free from contaminating DNA.

41. The method of embodiment 40, wherein the harvested broth is subjected to a broth conditioning process in the presence of a DNase preparation comprising one or more DNase proteins having at least 85% identity to SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17.

42. The method of embodiment 41, wherein the conditioned broth is subjected to a protein recovery process in the presence of a DNase preparation comprising one or more DNase proteins having at least 85% identity to SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17.

43. The method of any one of embodiments 40-42, wherein the one or more DNase proteins are protease resistant.

44. A protein preparation comprising a protein of interest (POI) produced according to the methods of any one of embodiments 40-42, wherein the protein preparation is essentially free of contaminating DNA.

45. A method for reducing the DNA content of a fermentation broth in which microbial host cells have been fermented comprising treating the broth with a DNase preparation comprising one or more proteins having at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17, wherein the treated broth is essentially free of contaminating DNA.

46. A method for reducing the DNA content of a recovered protein of interest (POI) preparation comprising treating the recovered POI preparation with a DNase preparation comprising one or more proteins having at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17, wherein the treated protein preparation is essentially free of contaminating DNA.

47. The method of any one of embodiments 36, 40, 44 or 45, wherein the microbial host is selected from a Gram negative bacterial cell, a Gram positive bacterial cell, a filamentous fungal cell, or a yeast cell.

48. The method of embodiment 47, wherein the Gram-negative bacterial host is an E. coli cell.

49. The method of embodiment 47, wherein the Gram-positive bacterial host is a Bacillus sp. cell.

50. The method of embodiment 47, wherein the filamentous fungal host is selected from an Aspergillus sp. cell, a Myceliophthora sp. cell and a Trichoderma sp. cell.

51. The method of embodiment 47, wherein the yeast cell is selected from a Yarrowia sp. cell or a Saccharomyces sp. cell.

52. The method of any one of embodiments 36, 40, or 46, wherein the POI is selected from the group consisting of a lyase, a ligase, a lectin, a hydrolase, an oxidoreductase, a transferase, an isomerase, an antibody, a receptor, and a cytokine.

53. The method of any one of embodiments 36, 40, or 46, wherein the POI is an animal feed protein or a food enzyme.

54. The method of embodiment 53, wherein the animal feed protein is selected from the group consisting of phytases, proteases, cellulases, 0-glucanases, xylanases, lipases, mannanases, α-galactosidases, pectinases and amylases.

55. The method of embodiment 53, wherein the food enzyme is selected from the group consisting of lactases, amylases, proteases, cellulases, lipases and xylanases.

### EXAMPLES

Certain aspects of the present disclosure may be further understood in light of the following examples, which should not be construed as limiting. Modifications to materials and methods will be apparent to those skilled in the art. Standard recombinant DNA and molecular cloning techniques used herein are well known in the art (Ausubel et al., 1987; Sambrook et al., 1989).

### Example 1

### Expression of Heterologous Bacterial Nucleases in Bacillus Cells

The present example describes the co-expression of certain prokaryotic DNases with an exemplary enzyme (i.e., FNA protease). More particularly, a parental Bacillus cell (CB455) expressing the FNA protease was modified to co-express a heterologous (foreign) DNase, such as modified Bacillus cells CB465 (expressing DNase TceNuc1) and CB467 (expressing DNase SdyNuc1) presented below in TABLE 1.

TABLE 1
Parental and Nuclease Modified Bacillus Strains

| Strain Name | Description |
|---|---|
| CB455 parent | FNA expression cassette |
| CB465 modified | FNA expression cassette + DNase TceNuc1 |
| CB467 modified | FNA expression cassette + DNase SdyNuc1 |

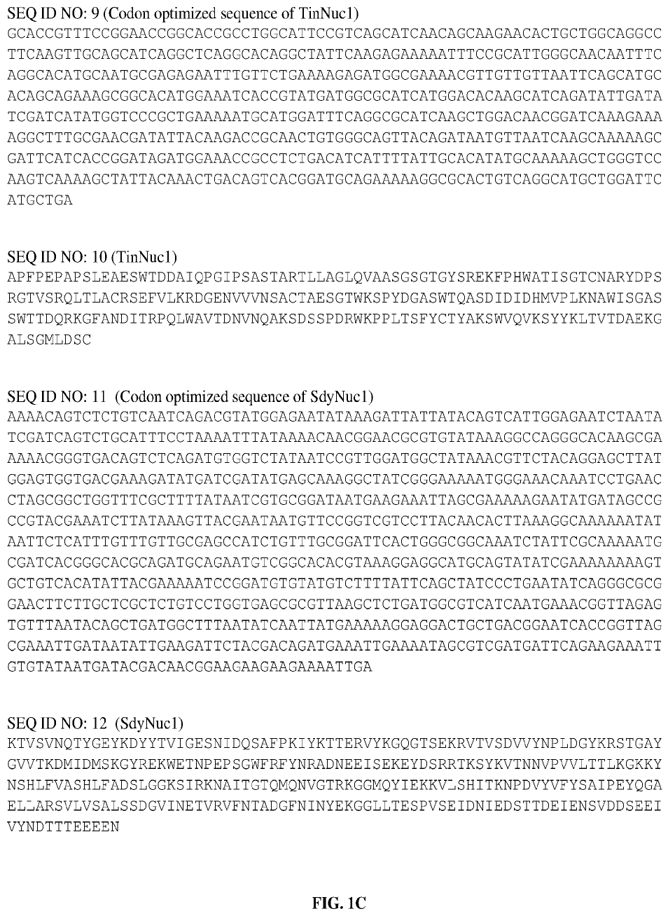

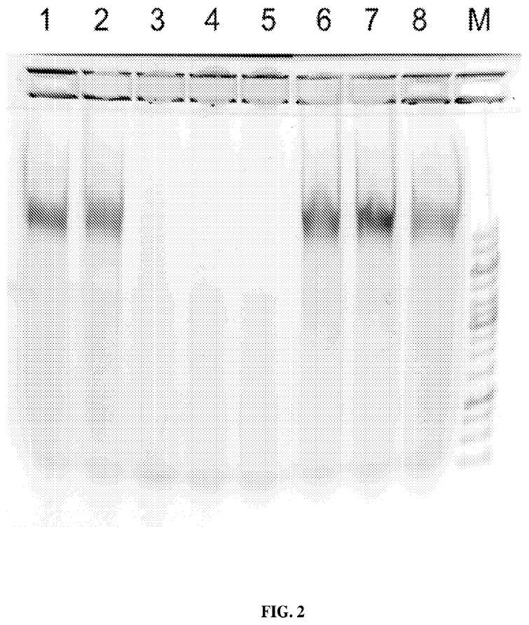

Any suitable promoter sequence and/or signal sequence operable in a Bacillus sp. cell may be used to express and/or secrete, respectively, the DNase of interest (e.g., TceNuc1, SdyNuc1). In the instant example, a B. subtilis aprE (gene) promoter sequence and its signal sequence were used for expression and secretion of the DNases, respectively. More particularly, the parental (CB455) and modified Bacillus cells expressing the DNase (TceNuc1 or SdyNuc1) were fermented in a twenty-four (24) well microtiter plate under identical conditions in a soytone based medium at 37° C. for forty-four (44) hours. For example, as presented in FIG. 2, the recombinant DNA (rDNA) released by lysed cells and present in the supernatants was visualized on E-Gel™ Agarose Gels, 0.8% (ThermoFisher catalogue No. G501808). Likewise, the rDNA present in the fermentation broth at forty-four (44) hours of fermentation (FIG. 2) was quantified using the Image Lab 6.0.1 software (BioRad), as shown below in TABLE 2.

TABLE 2
Results of Recombinant DNA (rDNA) Quantification

| (Lane No.) | Adj. Total Band | Total Band |
|---|---|---|
| Strain | Vol. (Int) | Vol. (Int) |
| (1) CB455 | 34,509,904 | 212,832,880 |
| (2) CB455 | 45,068,624 | 245,132,959 |
| (3) CB465 | 22,184 | 6,445,016 |
| (4) CB465 | 13,875 | 7,273,164 |
| (5) CB465 | 44,187 | 6,798,000 |
| (6) CB467 | 35,188,087 | 270,153,507 |
| (7) CB467 | 43,766,028 | 271,925,532 |
| (8) CB467 | 31,191,576 | 254,427,784 |

For example, TABLE 2 above shows the adjusted total band volumes after background removal and the total band volumes before background removal, wherein the parental strain CB455 (rows 1 and 2) and modified CB467 strain (rows 6-8) expressing the nuclease SdyNuc1, have highest amount of residual DNA, whereas the modified strain CB465 (rows 3-5) expressing nuclease TceNuc1 has the lowest amount of residual DNA. These rDNA quantification results demonstrate that the DNase TceNuc1 effectively degrades the rDNA present in the supernatant of the cell cultures, e.g., due to cell lysis.

### Example 2

### Expression of Heterologous Eukaryotic Nucleases in Bacillus Cells

The present example describes the co-expression of certain eukaryotic DNases with an exemplary enzyme (i.e., FNA protease). More particularly, a parental Bacillus cell (CB455) expressing the FNA protease was modified to co-express a heterologous DNase, such as modified Bacillus cells CB472, CB473, CB474 and CB475, presented below in TABLE 3.

TABLE 3
Parental and Nuclease Modified Bacillus Strains

| Strain Name | Description |
|---|---|
| CB455 parent | FNA expression cassette |
| CB472 modified | FNA expression cassette + DNase TreNuc1 |
| CB473 modified | FNA expression cassette + DNase BdeNuc1 |
| CB474 modified | FNA expression cassette + DNase GteNuc1 |
| CB475 modified | FNA expression cassette + DNase TinNuc1 |

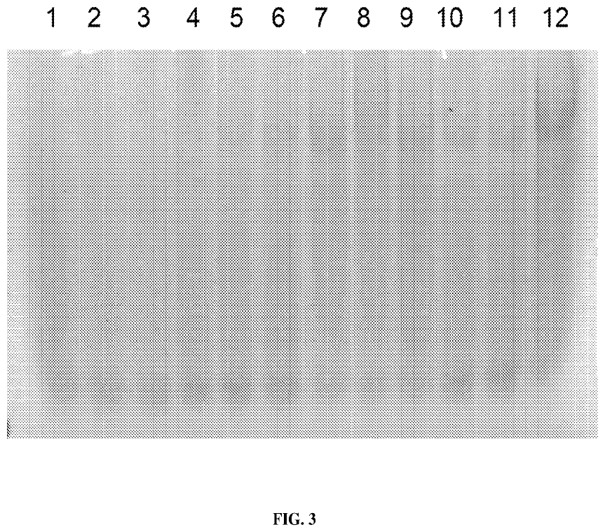

Any suitable promoter sequence and/or signal sequence operable in a Bacillus sp. cell may be used to express and secrete a nuclease of interest (e.g., TreNuc1, BdeNuc1, GteNuc1 and TinNuc1). In the instant example, a B. subtilis aprE (gene) promoter sequence and its signal sequence were used for expression and secretion of the nuclease, respectively. More particularly, the parental (CB455) and modified Bacillus cells expressing a DNase (TreNuc1, BdeNuc1, GteNuc1 or TinNuc1) were fermented in a twenty-four (24) well microtiter plate under identical conditions in a soytone based medium at 37° C. for forty-four (44) hours. For example, as presented in FIG. 3, the recombinant DNA (rDNA) released by lysed cells and present in the supernatants was visualized on E-Gel™ Agarose Gels (0.8%; ThermoFisher Catalogue No. G501808). The rDNA present in the fermentation broth at forty-four (44) hours of fermentation was quantified using the Image Lab 6.0.1 software (BioRad), as presented below in TABLE 4.

TABLE 4
Results of Recombinant DNA (rDNA) Quantification

| (Lane No.) | Adj. Total Band | Total Band |
|---|---|---|
| Strain | Vol. (Int) | Vol. (Int) |
| (1) CB472 | 25,056 | 2,531,712 |
| (2) CB472 | 13,833 | 2,295,321 |
| (3) CB472 | 6,351 | 2,236,248 |
| (4) CB473 | 882,006 | 23,566,908 |
| (5) CB473 | 2,437,724 | 50,916,112 |
| (6) CB473 | 3,267,894 | 57,269,142 |
| (7) CB474 | 5,873,109 | 60,207,480 |
| (8) CB474 | 5,034,081 | 61,060,167 |
| (9) CB474 | 6,122,886 | 64,823,961 |
| (10) CB475 | 3,603,888 | 42,533,169 |
| (11) CB475 | 3,562,880 | 51,353,580 |
| (12) CB455 | 13,720,683 | 90,955,194 |

For example, TABLE 4 above shows the adjusted total band volumes after background removal and the total band volumes before background removal. More particularly, as presented in TABLE 4, modified strain CB472 (rows 1-3) expressing nuclease TreNuc1, modified strain CB473 (rows 4-6) expressing nuclease BdeNuc1, modified strain CB474 (rows 7-9) expressing nuclease GteNuc1 and modified strain CB475 (rows 10 and 11) expressing nuclease TinNuc1, have lower amount of rDNA in their supernatants compared to the parental strain CB455 (row 12), demonstrating that the nucleases TreNuc1, BdeNuc1, GteNuc1 and TinNuc1 are particularly suitable for degrading residual DNA present in the supernatants of the cultures due to cell lysis.

### Example 3

### Recombinant Expression of Nucleases in Filamentous Fungal Cells

In the instant example, expression cassettes were developed for two wild-type fungal nucleases, TRENUC1 and TinNuc1. These expression cassettes were then used to transform an exemplary filamentous fungal strain (e.g., Trichoderma reesei), wherein transformants were subsequently screened in microtiter plate fermentations for the presence of the recombinant nucleases in fermentation broth by SDS-PAGE analysis. Additionally, Trichoderma strains expressing each nuclease individually were fermented in two (2) L fermentors/bioreactors, wherein the fermentation broth rapidly degraded DNA.

### A. Trichoderma Strains for the Expression of Nucleases

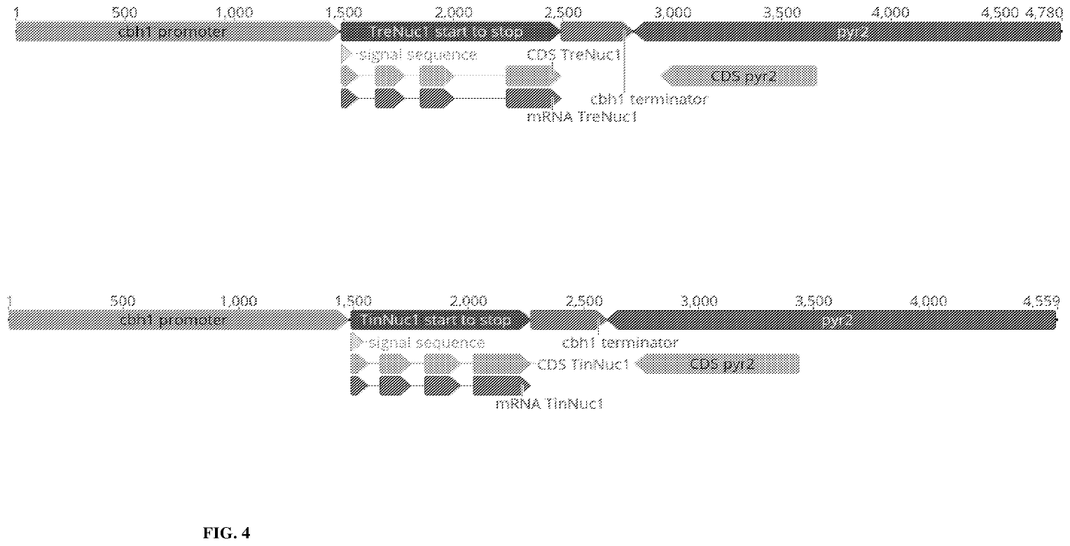

To generate expression cassettes for the TreNuc1 and TinNuc1 nucleases, the wild-type DNA sequences from the native source organisms from translational start to stop, were subcloned under the control of the Trichoderma reesei cbh1 promoter and terminator sequences, and linked to the Trichoderma pyr2 marker. For example, schematic maps of the expression cassettes constructed are shown in FIG. 4, wherein the TreNuc1 cassette (FIG. 4, top diagram) comprises SEQ ID NO: 13, and the TinNuc1 cassette (FIG. 4, bottom diagram) comprises SEQ ID NO: 14. As presented in FIG. 4, the TreNuc1 and TinNuc1 cassettes and/or additional expression cassettes are readily generated or synthesized by one skilled in the molecular biology arts (e.g., using routine molecular biology methods) and the detailed descriptions and examples provided herein.

The TreNuc1 and TinNuc1 cassettes were independently used to co-transform a Trichoderma strain along with Cas9 nuclease and synthetic guide RNAs (sgRNA) that targeted various regions within Trichoderma genome either individually or in combinations. These Cas9-sgRNA complexes were assembled in vitro according the manufacturer's protocol (Synthego) and used to transform Trichoderma strain as generally set forth in PCT Publication No. WO2016/100568 (incorporated herein by reference in its entirety). Approximately fifteen (15) μg of purified fragment and the assembled Cas9-sgRNA complex were used to transform protoplasts of a pyr2 mutant Trichoderma reesei strain wherein the four native cellulase genes, cbh1, cbh2, egl1 and egl2, had been deleted. The transformation was performed using the polyethylene glycol (PEG) mediated protoplast transformation protocol (Ouedraogo et al., 2015; Penttila et al., 1987). The transformants were grown on Vogel's minimal medium agar plates to select for uridine prototrophy acquired by the pyr2 marker. Transformants were then isolated and outgrown on Vogel's minimal agar plates before screening for nuclease expression.

### B. Expression of Nucleases in Microtiter Plates

The following section demonstrates that transformants expressing nucleases can be identified by fermentation in microtiter plates and analysis of the cell-free filtrates by SDS-PAGE.

Media composition: 400× T. reesei trace elements: citric Acid (anhydrous), 175 g/L; FeSO₄·7 H₂O, 200 g/L, ZnSO₄·7 H₂O, 16 g/L, CuSO₄·5 H₂O, 3.2 g/L; MnSO₄·H₂O, 1.4 g/L; H₃BO₃, 0.8 g/L.

Citrate minimal medium 5 g/L (NH₄)2504, 4.5 g/L KH₂PO₄, 1 g/L Mg504.7 H20, and 14.4 g/L citric acid, adjusted to pH 5.5 with 5% NaOH. After autoclaving for 30 minutes, sterile 50% glucose was added to a final concentration of 0.5%, along with 2.5 mL/L of 400× trace element solution.

Liquid defined (LD) culture medium contained the following components. Casamino acids, 9 g/L; (NH₄)₂SO₄, 5 g/L; MgSO₄·7H₂O, 1 g/L; KH₂PO₄, 4.5 g/L; CaCl₂·2H₂O, 1 g/L, PIPPS, 33 g/L, 400× T. reesei trace elements, 2.5 ml/L; pH adjusted to 5.5 with NaOH. After sterilization, lactose or a glucose/sophorose mixture was added to a final concentration of 1.6% w/v.

Production evaluation: Transformants and a spontaneous auxotroph of the parental strain were grown in citrate minimal media for 36-48 hours at 32° C. in 96 well plates with shaking. After incubation, 0.11 mL of seed culture were added to 0.99 ml of LD medium per well of a 24-well 20% lactose slow release micro-titer plate (srMTP). The lactose srMTPs have been described in US Patent Publication No. US20150147768A1 (incorporated herein by reference in its entirety). These production cultures were then fermented for four (4) to five (5) days at 25° C. and 250 RPM. Following fermentation, secreted proteins were separated from the cell mass by filtration through a filter-bottom 96-well plate into a 96-well non-binding assay plate. One (1) to three (3) microliters (μL) of filtrate was then evaluated by SDS-PAGE along with the See Blue Plus 2 molecular weight standard, followed by staining and detaining of the gel using standard molecular biology procedures.

As shown in FIG. 5, a new band of apparent molecular weight between 14 and 28 kDa was seen in the filtrate of transformants (e.g., lanes 3-6 for TreNuc1 and lanes 9-12 for TinNuc1), but not in that of a pyrimidine prototrophic derivative of the same parental strain (e.g., lanes 2, 7, 8 and 13). This is consistent with the calculated molecular weights of both nucleases based on primary amino acid sequences at 20 kDa (TreNuc1, SEQ ID NO: 4 and TinNuc1, SEQ ID NO: 17).

### C. Expression of Nucleases by Trichoderma in Bioreactor Scale (2L) Fermentors

The following section demonstrates that the small scale nuclease fermentations describe above (Section B) can be scaled up to two (2) liter bioreactors.

Fermentation: Briefly, spores of each strain were added separately to 50 mL of citrate minimal medium in a 250 ml flask with both side and bottom baffles. The cultures were grown for 48 hours at 28-30° C. at 170-240 RPM in a shaking incubator. After 48 hours, the contents of each flask were added separately to 2 L fermenters (bioreactors) for inoculation. Prior to inoculation, medium containing 0.95 kg of medium containing 4.7 g/L KH₂PO₄, 1.0 g/L MgSO₄·7·H₂O, 9 g/L (NH₄)2SO₄ and 2.5 mL/L trace element solution were added to the 2 L bioreactors and then heat sterilized together at 121° C. for 30 minutes. Post-sterile additions were added before inoculation to the tank: sterilized 4.8 mL of 50% glucose and 0.96 mL of 0.48% CaCl₂·2H₂O. The medium was adjusted to pH 3.5 with 14% NH₃ and the temperature was maintained at 30° C., and the pH at 3.5 for the entire growth period.

A dissolved oxygen (DO) probe was calibrated to 100% when there was no added pressure in the headspace (i.e., 0 bar gauge, 1 bar absolute). The bioreactor contained a four-blade Rushton impeller in between two marine impellers, which provided mixing via a variable speed motor that was initially set at 800 RPM. The control of dissolved oxygen inside the bioreactor was based on a control loop adjusting several set-points. When DO fell below 30%, the agitation rate was increased to maintain the dissolved oxygen at 30%, with a maximum agitation set point of 1,200 RPM. If the DO could not hold at 30%, oxygen enrichment was increased from 21% to up to 40%. If the DO still could not hold, then the gas flow was increased from 60 sL/h to up to 80 sL/h.

Strains completely consumed glucose and reached substantially the same biomass concentration (about 40 g/kg dry cell weight) at around the same time (about 30 hours into the fermentation). Following exhaustion of batched glucose, pH was adjusted and maintained at about 5 with ammonia, the temperature was lowered to 25° C. and a slow feed of glucose and mixed disaccharides was started to maintain the cells in a glucose-limited state and encourage protein production. At various timepoints fermentation samples were extracted from the bioreactor and frozen at −20C. Following competition of the fermentation, thawed whole cell broth was transferred to a microcentrifuge tube and the cells pelleted by centrifugation. The supernatant containing the secreted proteins was evaluated by SDS-PAGE analysis using standard molecular biology techniques.

As shown in FIG. 6, both nucleases (TreNuc1 and TinNuc1) expressed well in the bioreactors. More particularly, as presented in FIG. 6, the TreNuc1 (nuclease) protein accumulated throughout the fermentation, whereas the TinNuc1 (nuclease) protein reaches a maximum concentration around 114 hours, but protein was lost in later fermentation time points.

### D. Degradation of DNA by Recombinant Nucleases Expressed by Trichoderma

The following section demonstrates that the expressed recombinant nucleases TreNuc1 and TinNuc1 can efficiently remove DNA from a concentrated Trichoderma whole cellulase product, Primafast® 200 (IFF).

Nuclease Assay: Supernatants as generated above in section C were used to treat a research grade preparation of commercial product Primafast® 200 (IFF), by mixing various components as outlined below in TABLE 5. For nuclease TreNuc1, supernatant from 186 hours of fermentation was used and for nuclease TinNuc1, supernatant from 114 hours of fermentation was used. Additional DNA was spiked into the product for some reactions, wherein the spiked DNA was genomic DNA (gDNA) from Trichoderma reesei at approximately 300 ng/μl. Samples were split 20 μl each to PCR strip tubes and incubated for four (4) hours with one strip at 4° C. and the other at room temperature (25° C.). Five (5) microliters of the reactions were loaded directly onto a double comb 0.8% eGel (Invitrogen) along with 1 Kb molecular weight marker (Invitrogen) and run for twelve (12) minutes.

TABLE 5
NUCLEASE ASSAY REACTION COMPONENTS

| | | | TreNuc1 | TinNuc1 | Nuclease- |
|---|---|---|---|---|---|
| Assay | Primafast | Genomic | Super- | Super- | free |
| Reaction | 200 | DNA | natant | natant | Water |
| No. | (μl) | (μl) | (μl) | (μl) | (μl) |
| 1 | 24 | 0 | 0 | 0 | 16 |
| 2 | 24 | 0 | 8 | 0 | 8 |
| 3 | 24 | 0 | 0 | 8 | 8 |
| 4 | 0 | 8 | 0 | 0 | 32 |
| 5 | 24 | 8 | 0 | 0 | 8 |
| 6 | 24 | 8 | 8 | 0 | 0 |
| 7 | 24 | 8 | 0 | 8 | 0 |

As shown in FIG. 7, without DNAse treatment a low amount of low molecular weight DNA was detected in Primafast (lane 1) with incubation at 4° C. (top) or 25° C. (bottom). With addition of broth from either TreNuc1 or TinNuc1 nuclease expressing strains, this band disappeared even with 4° C. incubation (lane 2 and 3, top). Since the amount of DNA in Primafast was low, Trichoderma gDNA was added to Primafast to 60 ng/μl to enable better visualization of nuclease action and to simulate a condition of high DNA contamination. This high molecular weight DNA shifted mobility after addition to Primafast (compare lane 5 to 4), perhaps due to the presence of salts in formulated Primafast product. At 25° C., some degradation of the DNA was seen, but most DNA was still high molecular weight (compare lane 5 top and bottom). This implies some nuclease was already present in the product, but was insufficient to eliminate contaminating DNA even at an elevated temperature of 25° C. (see lanes 1 and 5, bottom). Thus, as shown in FIG. 7, with the addition of broth from either TreNuc1 or TinNuc1 nuclease expressing strains, all high molecular weight DNA was eliminated at either incubation temperature tested (lanes 7 and 8).

### Example 4

Nuclease Treatments of Whole Fermentation Broths, Supernatants and/or Protein Preparations Obtained Therefrom

The instant example generally describes nuclease treatments suitable for use on whole fermentation broths, supernatants, protein preparations, isolated proteins, concentrated proteins and the like, using the TreNuc1 nuclease as an exemplary DNase. More specifically, the TreNuc1 nuclease treatment described herein was performed on an ultrafiltration concentrate (UFC) obtained from the fermentation of a Bacillus subtilis strain over-expressing a heterologous Pseudomonas sp. lipase. Thus, a UFC derived from a T. reesei strain expressing the TreNuc1 nuclease (Example 3) was added to the lipase (polyesterase) UFC unformulated or formulated with 45% Glycerol, pH 6.5. at concentrations (v/v) of 0.1% and 1%, and incubated overnight at 4° C. or at room temperature (25° C.). After the incubation, the presence of recombinant DNA was detected by direct visualization of one (1) μl of treated and untreated UFC samples on agarose gel or by PCR amplification of a fragment within the polyesterase gene and visualization of the PCR reactions on an agarose gel. For the PCR amplification, one (1) μl of 4× diluted sample was used in the PCR reaction using Q5 polymerase. TABLE 6 set forth below describes the samples/conditions as loaded in the agarose gel, and shown in FIG. 9 (lanes 1-8)

TABLE 6
SAMPLES LOADED ON AGAROSE GEL AND USED FOR THE
PCR REACTION ON THE POLYESTERASE GENE (rDNA)

| Lane No. | Sample Description |
|---|---|
| 1 | Unformulated (UN) lipase sample incubated |
| | at 4° C., no nuclease treatment |
| 2 | Unformulated (UN) lipase sample incubated at |
| | 4° C. treated with 0.1% UFC TreNuc1 nuclease |
| 3 | Unformulated (UN) lipase sample incubated at |
| | 4° C. treated with 1% UFC TreNuc1 nuclease |
| 4 | Formulated (F) lipase sample incubated at |
| | 4° C., no nuclease treatment |
| 5 | Formulated (F) lipase sample incubated at |
| | 4° C. treated with 0.1% UFC TreNuc1 nuclease |
| 6 | Formulated (F) lipase sample incubated at |
| | 4° C. treated with 1% UFC TreNuc1 nuclease |
| 7 | Formulated (F) lipase sample incubated at |
| | 25° C. treated with 0.1% UFC TreNuc1 nuclease |
| 8 | Formulated (F) lipase sample incubated at |
| | 25° C. treated with 1% UFC TreNuc1 nuclease |

More particularly, FIG. 9 shows an agarose gel image of the unformulated (UN) and formulated (F) polyesterase samples described above in TABLE 6 (i.e., before and after treatment of with TreNuc1 nuclease UFC), and FIG. 10 shows an agarose gel image of the DNA fragments amplified by PCR using oligonucleotides that amplify a specific sequence within the polyesterase gene of the samples described above in TABLE 6 (i.e., before and after treatment of with TreNuc1 nuclease UFC).

In FIG. 9, the recombinant DNA (rDNA) released by lysed cells and present in the UFC of the lipase sample was visualized on E-Gel™ Agarose Gels (0.8%; ThermoFisher Catalogue No. G501808). Lanes 1 and 4 show the untreated formulated and unformulated lipase samples. Lanes 2 and 3 show the unformulated samples treated with 0.1% or 1% of TreNuc1 UFC incubated at 4° C. Lanes 5 and 6 show the formulated samples treated with 0.1% or 1% of TreNuc1 UFC incubated at 4° C. Lane 7 and 8 show the formulated samples treated with 0.1% or 1% of TreNuc1 UFC incubated at room temperature. The disappearance of the lower molecular weight band in the samples treated with 0.1% and 1% of TreNuc1 nuclease is indicative of DNase activity in the TreNuc1 UFC.

Demonstration of DNase activity in the TreNuc1 UFC is shown in FIG. 10. The lipase UFC samples untreated and treated with 0.1% and 1% of TreNuc1 UFC were used in a PCR reaction targeting the amplification of a 320 bp DNA fragment within the lipase coding sequence. The results were visualized on E-Gel™ Agarose Gels (0.8%; ThermoFisher Catalogue No. G501808). The agarose gel in FIG. 10 shows on left the TriDye™ 1 kb Plus DNA Ladder (L); lane 1 and 4 show the amplified 320 bp DNA fragment using the unformulated or formulated lipase samples as templates in the PCR reactions, lanes 2 and 3 show the results of the PCR reactions on the unformulated samples after treatment with 0.1% and 1% TreNuc1 UFC at 4° C.; lanes 5 and 6 show the results of the PCR reactions on the formulated samples after treatment with 0.1% and 1% TreNuc1 UFC at 4° C. and lanes 7 and 8 show the results of the PCR reactions on the formulated samples after treatment with 0.1% and 1% TreNuc1 UFC at room temperature. The absence of an amplified PCR product in the samples treated with TreNuc1 UFC demonstrated that the DNase activity of TreNuc1 UFC can effectively degrade rDNA present in the lipase samples.

### REFERENCES

PCT Publication No. WO1989/06279
PCT Publication No. WO1990/11352
PCT Publication No. WO1994/18314
PCT Publication No. WO1999/050389
PCT Publication No. WO1999/19467
PCT Publication No. WO1999/20726
PCT Publication No. WO1999/20769
PCT Publication No. WO1999/20770
PCT Publication No. WO1999/43794
PCT Publication No. WO2000/29560
PCT Publication No. WO2000/60059
PCT Publication No. WO2003/076580
PCT Publication No. WO2006/037483
PCT Publication No. WO2006/037484
PCT Publication No. WO2006/089107
PCT Publication No. WO2008/063109
PCT Publication No. WO2008/065200
PCT Publication No. WO2008/112459
PCT Publication No. WO2011/010094
PCT Publication No. WO2011/072099
PCT Publication No. WO2013/043860
PCT Publication No. WO2014/164777
PCT Publication No. WO2016/100568
PCT Publication No. WO2019/081721
U.S. Pat. No. 4,914,031
U.S. Pat. No. 4,980,288
U.S. Pat. No. 5,208,158
U.S. Pat. No. 5,310,675
U.S. Pat. No. 5,336,611
U.S. Pat. No. 5,399,283
U.S. Pat. No. 5,441,882
U.S. Pat. No. 5,482,849
U.S. Pat. No. 5,631,217
U.S. Pat. No. 5,665,587
U.S. Pat. No. 5,700,676
U.S. Pat. No. 5,741,694
U.S. Pat. No. 5,858,757
U.S. Pat. No. 5,880,080
U.S. Pat. No. 6,197,567
U.S. Pat. No. 6,218,165
U.S. Pat. No. 6,255,115
US Patent Publication No. US2012/013549
US Patent Publication No. US2015/0147768
Ausubel et al., “Current Protocols in Molecular Biology”, published by Greene Publishing Assoc. and Wiley-Interscience (1987).
EFSA, “Guidance on the characterisation of microorganisms used as feed additives or as production organisms”, EFSA Journal 16(3):5206, 2018.
Kriegler, “Gene Transfer and Expression: A Laboratory Manual”, 1990.
Ouedraogo et al., “I-SceI-mediated double-strand DNA breaks stimulate efficient gene targeting in the industrial fungus Trichoderma reesei”, Appl. Microbiol. Biotechnol., 99 (pages 10083-10095), 2015.
Penttila et al., “A versatile transformation system for the cellulolytic filamentous fungus Trichoderma reesei”, Gene (Amst.) 61, 155-164, 1987.
Sambrook et al., “Molecular Cloning: A Laboratory Manual” Cold Spring Harbor Laboratory: Cold Spring Harbor, N.Y. (1989), (2001) and (2012).
Wu et al., “Structures, Mechanisms, and Functions of His-Me Finger Nucleases”, Trends in Biochemical Sciences, 45(11): 935-946, 2020.

1. An isolated nucleic acid comprising at least 80% identity to a nucleotide sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO:14 and SEQ ID NO:15.

2. An isolated nucleic acid encoding a protein comprising at least 85% identity to a protein selected from the group consisting of SEQ ID NO: 2, SEQ ID NO: 4, SEQ ID NO: 6, SEQ ID NO: 8, SEQ ID NO: 10, SEQ ID NO: 12, SEQ ID NO:16 and SEQ ID NO: 17.

3. (canceled)

4. A vector comprising a nucleic acid of claim 1.

5. A recombinant microbial cell comprising one or more introduced vectors of claim 4.

6. An isolated protein comprising at least 85% identity to a protein selected from the group consisting of SEQ ID NO: 2, SEQ ID NO: 4, SEQ ID NO: 6, SEQ ID NO: 8, SEQ ID NO: 10, SEQ ID NO: 12, SEQ ID NO:16 and SEQ ID NO: 17, wherein the protein comprises deoxyribonuclease (DNase) activity.

7-8. (canceled)

9. A protein preparation comprising one or more proteins of claim 6.

10. A recombinant microbial cell expressing one or more proteins comprising at least 85% identity to a protein sequence of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17; or a recombinant microbial cell co-expressing a protein of interest (POI) and one or more proteins comprising at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17.

11. The recombinant cell of claim 10, selected from a Gram-negative bacterial cell, a Gram-positive bacterial cell, a filamentous fungal cell, or a yeast cell.

12. A fermentation broth obtained by fermenting a microbial cell expressing one or more proteins comprising at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17; or a fermentation broth obtained by fermenting a microbial cell co-expressing (i) one or more proteins of interest and (ii) one or more proteins comprising at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17.

13. The broth of claim 12, wherein the one or more proteins of interest isolated from the broth are essentially free of contaminating DNA.

14. A method for producing a protein of interest (POI) essentially free from contaminating DNA comprising:
(a) obtaining or constructing a microbial cell expressing a POI and modifying the cell to express one or more DNase proteins comprising at least 85% identity to a protein of SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17.
(b) fermenting the modified cell under suitable conditions for the expression of the POI and one or more DNase proteins, and
(c) harvesting the fermentation broth at the end of fermentation,
wherein the POI isolated from the harvested broth is essentially free from contaminating DNA.

15. A method for producing a protein of interest (POI) essentially free from contaminating DNA comprising:
(a) obtaining or constructing a microbial cell expressing a POI and fermenting the cell under suitable conditions for the expression of the POI, and
(b) harvesting the fermentation broth at the end of fermentation, wherein the harvested broth is treated with a DNase preparation comprising one or more DNase proteins having at least 85% identity to SEQ ID NO: 2, SEQ ID NO: 4, SEQ ID NO: 6, SEQ ID NO: 8, SEQ ID NO: 10, SEQ ID NO: 12, SEQ ID NO:16 or SEQ ID NO: 17.
wherein the DNase treated broth is essentially free from contaminating DNA.


SEQUENCE LISTING (17 sequences)

SEQ ID NO: 1 - DNA, 630 bp, Thermobifida cellulosilytica
  source 1..630: mol_type = other DNA
ttagatgatttgttagcggaactggaagaaattgaagcggatgtgcctgcgacagcggaatcacaagcacctgaaggagatgcggatgcagtcgaagcacgtcaacgcttaggagaaatccctatcgcagaaccggcgcctagaggagattatgatagagatgaatttggctcaggctggatcgatacggatggcaatggctgctctacgcgtaatgatattcttgcacgcgatcttgaaaatgtccaacttgcgtctgatggctgcaaagtgttgtctggaacacttcatgatccgtatacaggcgcgacgattgaatttacatcagaaaaacctcaagcagttcaaatcgatcatgtggttccgttgtcacttgcgtggcgcatgggagcatctcggtgggatcgtgatacacgggtcgaatttgcaaatgatccgggcaatctgttagcgtcagatggcccggcgaatagagaaaaatcagattctggaccgggcgaatggcaaccttatgaaggctttcaatgcacatatgcagtcctgtatattgatgtgctgtatcgctatgatttaccggtgagcgaacaagattatgaaggactgagcgcaatgttggatacgtgcgcatga

SEQ ID NO: 2 - protein, 209 aa, Thermobifida cellulosilytica
LDDLLAELEEIEADVPATAESQAPEGDADAVEARQRLGEIPIAEPAPRGDYDRDEFGSGWIDTDGNGCSTRNDILARDLENVQLASDGCKVLSGTLHDPYTGATIEFTSEKPQAVQIDHVVPLSLAWRMGASRWDRDTRVEFANDPGNLLASDGPANREKSDSGPGEWQPYEGFQCTYAVLYIDVLYRYDLPVSEQDYEGLSAMLDTCA

SEQ ID NO: 3 - DNA, 561 bp, Hypocrea jecorina
  source 1..561: mol_type = other DNA
gcaccgttacctgcaccgcctggcattccgtcagaagatacagcaagaacacaactggcaggccttacagttgcagttgttggctcaggcacaggctattcaagagatctttttccgacatgggatgcaatttcaggcaattgcaatgcaagagaatatgttctgaaaagagatggcgaaggcgtccaagttaataatgcatgcgaagcacaatcaggctcatggatttcaccgtatgataatgcgtcatttacaaatgcaagcagcctggatattgatcatatggttccgctgaaaaatgcgtggatttcaggcgcatcaacatggacaacagcacaacgcgaagcactggcaaatgatgtttcaagaccgcaactgtgggcagtttcagcatcatcaaatagatcaaaaggcgatagatcaccggatcaatggaaaccgcctctgacatcattttattgcacatatgcaaaaagctggatcgacgtcaaaagctattacaaactgacaatcacgagcgcagaaaaaacagcactgtcatcaatgctggatacatgctga

SEQ ID NO: 4 - protein, 186 aa, Hypocrea jecorina
APLPAPPGIPSEDTARTQLAGLTVAVVGSGTGYSRDLFPTWDAISGNCNAREYVLKRDGEGVQVNNACEAQSGSWISPYDNASFTNASSLDIDHMVPLKNAWISGASTWTTAQREALANDVSRPQLWAVSASSNRSKGDRSPDQWKPPLTSFYCTYAKSWIDVKSYYKLTITSAEKTALSSMLDTC

SEQ ID NO: 5 - DNA, 573 bp, Blastomyces gilchristii
  source 1..573: mol_type = other DNA
tcaacaatcccgctgcataaaagagatccgcctaatattccgtcaccgcctgaagcatcaagactgctggaactgatggttgtttcaccgcaaggaccgcaaacaggctattcaagaacaaaatttccgcattggatcacaattaacggaccgtgcaatacaagagaaacagttctgaaacgtgatggcatgaatgttacagttggcaaaaattgctcaccgaaatcaggctcatggtattcaacatatgatggcgttacactgacaagctcaagcaatgttgatattgatcatgttgttccgctgtcagatgcatggaaatctggcgcatcaggctggacaagagaacgtagaagagattttgcaaacgatatggaaaatccgcaactgatttcagttcagggcaaagtcaataaagaaaagggcgataaaggaccggataaatggaaaccgcctctgccgacatatcattgcacatatgcgtgcatgtgggttaaagttaaaagcgcgtataacctgacaatcacggatccggaaaaacaagcactgatggatatgctgggcacatgctga

SEQ ID NO: 6 - protein, 190 aa, Blastomyces gilchristii
STIPLHKRDPPNIPSPPEASRLLELMVVSPQGPQTGYSRTKFPHWITINGPCNTRETVLKRDGMNVTVGKNCSPKSGSWYSTYDGVTLTSSSNVDIDHVVPLSDAWKSGASGWTRERRRDFANDMENPQLISVQGKVNKEKGDKGPDKWKPPLPTYHCTYACMWVKVKSAYNLTITDPEKQALMDMLGTC

SEQ ID NO: 7 - DNA, 567 bp, Gelasinospora tetrasperma
  source 1..567: mol_type = other DNA
gcaccggcaccgatgcctacaccgcctggcattccgacagcagcaaatgcaaaaacacaactggcaagcctgacagttaaagcatatgttgatgacggcaaatatgaacgcgatctttttccgacatggattacaatttcaggcacatgcaatacacgcgaatatgttctgaaaagagatggcacaaacgttgttacagattcagcatgcacagcaacaagcggcacatgggcatcaccgtatgatggcggagtttggacagcagcgtcagatgttgatattgatcatatggttccgctgaaaaatgcatgggtttcaggcgcaaatgcgtggacaacatcaaaaagacaacaatttgcgaacgacattaattcaccgcaactgtgggcagttacagataatgttaatcaagcgaaaagcgatcaatcaccggatgcatggaaaccgcctctgacatcattttattgcacatatgcaaaagcatgggtccaagtcaaatatacatgggcactgacaattacgagcgcagaaaaatcagcactgacatcaatgctgaatacgtgctga

SEQ ID NO: 8 - protein, 188 aa, Gelasinospora tetrasperma
APAPMPTPPGIPTAANAKTQLASLTVKAYVDDGKYERDLFPTWITISGTCNTREYVLKRDGTNVVTDSACTATSGTWASPYDGGVWTAASDVDIDHMVPLKNAWVSGANAWTTSKRQQFANDINSPQLWAVTDNVNQAKSDQSPDAWKPPLTSFYCTYAKAWVQVKYTWALTITSAEKSALTSMLNTC

SEQ ID NO: 9 - DNA, 567 bp, Tolypocladium inflatum
  source 1..567: mol_type = other DNA
gcaccgtttccggaaccggcaccgcctggcattccgtcagcatcaacagcaagaacactgctggcaggccttcaagttgcagcatcaggctcaggcacaggctattcaagagaaaaatttccgcattgggcaacaatttcaggcacatgcaatgcgagagaatttgttctgaaaagagatggcgaaaacgttgttgttaattcagcatgcacagcagaaagcggcacatggaaatcaccgtatgatggcgcatcatggacacaagcatcagatattgatatcgatcatatggtcccgctgaaaaatgcatggatttcaggcgcatcaagctggacaacggatcaaagaaaaggctttgcgaacgatattacaagaccgcaactgtgggcagttacagataatgttaatcaagcaaaaagcgattcatcaccggatagatggaaaccgcctctgacatcattttattgcacatatgcaaaaagctgggtccaagtcaaaagctattacaaactgacagtcacggatgcagaaaaaggcgcactgtcaggcatgctggattcatgctga

SEQ ID NO: 10 - protein, 188 aa, Tolypocladium inflatum
APFPEPAPPGIPSASTARTLLAGLQVAASGSGTGYSREKFPHWATISGTCNAREFVLKRDGENVVVNSACTAESGTWKSPYDGASWTQASDIDIDHMVPLKNAWISGASSWTTDQRKGFANDITRPQLWAVTDNVNQAKSDSSPDRWKPPLTSFYCTYAKSWVQVKSYYKLTVTDAEKGALSGMLDSC

SEQ ID NO: 11 - DNA, 879 bp, Streptococcus dysgalactiae
  source 1..879: mol_type = other DNA
aaaacagtctctgtcaatcagacgtatggagaatataaagattattatacagtcattggagaatctaatatcgatcagtctgcatttcctaaaatttataaaacaacggaacgcgtgtataaaggccagggcacaagcgaaaaacgggtgacagtctcagatgtggtctataatccgttggatggctataaacgttctacaggagcttatggagtggtgacgaaagatatgatcgatatgagcaaaggctatcgggaaaaatgggaaacaaatcctgaacctagcggctggtttcgcttttataatcgtgcggataatgaagaaattagcgaaaaagaatatgatagccgccgtacgaaatcttataaagttacgaataatgttccggtcgtccttacaacacttaaaggcaaaaaatataattctcatttgtttgttgcgagccatctgtttgcggattcactgggcggcaaatctattcgcaaaaatgcgatcacgggcacgcagatgcagaatgtcggcacacgtaaaggaggcatgcagtatatcgaaaaaaaagtgctgtcacatattacgaaaaatccggatgtgtatgtcttttattcagctatccctgaatatcagggcgcggaacttcttgctcgctctgtcctggtgagcgcgttaagctctgatggcgtcatcaatgaaacggttagagtgtttaatacagctgatggctttaatatcaattatgaaaaaggaggactgctgacggaatcaccggttagcgaaattgataatattgaagattctacgacagatgaaattgaaaatagcgtcgatgattcagaagaaattgtgtataatgatacgacaacggaagaagaagaaaattga

SEQ ID NO: 12 - protein, 292 aa, Streptococcus dysgalactiae
KTVSVNQTYGEYKDYYTVIGESNIDQSAFPKIYKTTERVYKGQGTSEKRVTVSDVVYNPLDGYKRSTGAYGVVTKDMIDMSKGYREKWETNPEPSGWFRFYNRADNEEISEKEYDSRRTKSYKVTNNVPVVLTTLKGKKYNSHLFVASHLFADSLGGKSIRKNAITGTQMQNVGTRKGGMQYIEKKVLSHITKNPDVYVFYSAIPEYQGAELLARSVLVSALSSDGVINETVRVFNTADGFNINYEKGGLLTESPVSEIDNIEDSTTDEIENSVDDSEEIVYNDTTTEEEEN

SEQ ID NO: 13 - DNA, 4770 bp, synthetic construct
  misc_feature 1..4770: note = synthetic cassette
  source 1..4770: mol_type = other DNA
tgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggacaccatgaagctgtctttctctattgccctcgcctcggccatcgcggctctcgctgctccggctcctctacctgcaccggtgcgtaacttctcctccgaccagtctcagcaccataaatctacatacacatcagatatactgacgacccaactgtaatagcccgggatcccatccgaagacacggcgagaacccagctcgccggcctcacagtcgccgttgttggttctggcacgggctactcccgcgacttgtttcctacctgggatgccatctccggcaactgcaatgctcggtacgtcaagcttccttggattcctactgataaaatacgaagaggctgactggcatattgccataacagcgagtacgtgttgaagcgagatggcgagggcgtccaggtcaacaatgcctgcgaggcccagtctgggagctggatcagcccctatgacaatgccagtttcacaaacgcgtccagcctggacattgaccacatggtgcctctgaagaatgcctggattgtgagtctgccatcttgcttctccgtggtctcagtctccatgtccctctctgtccatcgttgccctctgatatacccctggaactgttttcacctctgcctcacacccacataacctcagcatctttgtcacactcatcacttcactacagttcttctacttactttattctccccttcgacctttctttccaccccctctcatctcatctcattacaccaactgactcgacccagtccggcgcctcaacctggaccaccgcccagcgcgaggccctcgccaacgacgtctcccgcccgcagctctgggccgtctccgcgagctccaaccgctccaagggcgaccgcagccccgaccagtggaagcccccgctgaccagcttctactgcacgtacgccaagtcgtggattgacgtcaagagctattacaagttgactattacgagcgcggagaagacggcgctgagcagcatgttggatacctgctagtaaatgctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcggctagctagttacgcttgtttatttacgacaagatctagaagattcgagatagaataataataataacaacaatttgcctcttctttccaccttttcagtcttactctcccttctgacattgaacgcctcaatcagtcagtcgccttgtacttggcacggtaatcctccgtgttcttgatatcctcaggggtagcaaagcccttcatgccatcgataatgtcatccagagtgaggatggcaaagatggggatgccgtactccttcctcagctcgccaatggcactcggtccaggcttggagtcgtcgccatccgcagcggggagcttctccatgcggtccagggccacgacgatgccggcgacgatgccgccctccttggtgatcttctcaatggcgtccctcttggcggtgccggcggtgatgacgtcgtcgacaatcaggaccctcttgcccttgagcgaagcgccgacgatgttgccgccctcgccgtggtccttggcctccttgcggtcaaacgagtaggagacgcggtccaggttctggggcgccagctcgccgagcttgatggtgatggcggagcacagcgggatgcccttgtaggccgggccgaagacgatgtcgaactctaggccggccttctcctgggcctcgatgatggtctttgcaaaggcggaggcgatggcgccggcgaggcgcgccgtgtggaattcgcccgcgttgaagaagtagggggatatccgcttggacttgagctcgaagctgccaaacttgaggacgccgccgtcgatggcggatttgaggaagtcctgcttgtaggcaggcagctgggaggtggtagccattctgttggatttggatagtgtccttattctctgatttgaacagtagatcaggacgagtgagagggatgcagaggttggattggagtggttgagctataaaatttagaggcgcgccgtatcgagttttcacatggaagtcaaagcgtacagtgcgagcttgtacgttggtcttagtatcccacaagcttctgtctaggtatgatgatggctataagtcacccaaggcagaactcatcttgaagattgtctagagtgattttaccgctgatgaaatgactggactccctcctcctgctcttatacgaaaaattgcctgactctgcaaaggttgtttgtcttggaagatgatgtgcccccccatcgctcttatctcataccccgccatctttctagattctcatcttcaacaagaggggcaatccatgatctgcgatccagatgtgcttctggcctcatactctgccttcaggttgatgttcacttaattggtgacgaattcagctgatttgctgcagtatgctttgtgttggttctttccaggcttgtgccagccatgagcgctttgagagcatgttgtcacctataaactcgagtaacggccacatattgttcactacttgaatcacatacctaattttgatagaattgacatgtttaaagagctgaggtagctttaatgcctctgaagtattgtgacacagcttctcacagagtgagaatgaaaagttggactccccctaatgaagtaaaagtttcgtctctgaacggtgaagagcatagatccggcatcaactacctggctagactacgacgtcaattctgcggccttttgacctttatatatgtccattaatgcaatagattcttttttttttttttttttttttttttttttttttgcccaatttcgcagatcaaagtggacgttatagcatcataactaagctcagttgctgagggaagccgtctactaccttagcccatccatccagctccataccttgatactttagacgcgaagcaattcacactgtacgtctcgcagctctccttcccgctcttgcttccccactggggtccatggtgcgtgtatcgtcccctccttaat

SEQ ID NO: 14 - DNA, 4549 bp, synthetic construct
  misc_feature 1..4549: note = synthetic cassette
  source 1..4549: mol_type = other DNA
tgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggacaccatgaagttctccgcctccgttctcctgtccgccgccgcggcggtcctggcagcacctttccctgagccggctcctgtaagcacccgtagtcactggaggctgagagctggactgacgacgcaatacagccgggtatccccagcgcttccaccgccaggacgctgctcgctggcctccaggttgccgcttcggggagcgggaccggctacagtcgcgagaagttcccccactgggcgaccatcagcggcacttgcaacgcccggtacgacccgtcgcgtggtaccgtctcacggcaagtagctgacactggcctgccgtagcgagtttgtgctgaagcgcgatggcgagaacgtcgtggtcaactcggcctgcaccgccgagtcgggcacctggaagagcccgtacgacggcgcctcctggactcaggccagcgacattgacattgaccacatggtccccctcaagaacgcctggatcgtgaggcctacctctccgtgcctcatatcttggaatcgtactgacttgtgttagtctggagcctcctcgtggaccacggaccagcgcaagggcttcgccaacgatattacgcgcccgcagctctgggccgtgaccgacaatgtcaaccaggccaagagcgactcgagccccgacaggtggaagccgccgctgacgagcttctactgcacctatgctaagagctgggtgcaggtgaagagctactacaagctgaccgtcaccgatgccgaaaagggggccctgtcaggcatgctggacagctgctgataaatgctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcggctagctagttacgcttgtttatttacgacaagatctagaagattcgagatagaataataataataacaacaatttgcctcttctttccaccttttcagtcttactctcccttctgacattgaacgcctcaatcagtcagtcgccttgtacttggcacggtaatcctccgtgttcttgatatcctcaggggtagcaaagcccttcatgccatcgataatgtcatccagagtgaggatggcaaagatggggatgccgtactccttcctcagctcgccaatggcactcggtccaggcttggagtcgtcgccatccgcagcggggagcttctccatgcggtccagggccacgacgatgccggcgacgatgccgccctccttggtgatcttctcaatggcgtccctcttggcggtgccggcggtgatgacgtcgtcgacaatcaggaccctcttgcccttgagcgaagcgccgacgatgttgccgccctcgccgtggtccttggcctccttgcggtcaaacgagtaggagacgcggtccaggttctggggcgccagctcgccgagcttgatggtgatggcggagcacagcgggatgcccttgtaggccgggccgaagacgatgtcgaactctaggccggccttctcctgggcctcgatgatggtctttgcaaaggcggaggcgatggcgccggcgaggcgcgccgtgtggaattcgcccgcgttgaagaagtagggggatatccgcttggacttgagctcgaagctgccaaacttgaggacgccgccgtcgatggcggatttgaggaagtcctgcttgtaggcaggcagctgggaggtggtagccattctgttggatttggatagtgtccttattctctgatttgaacagtagatcaggacgagtgagagggatgcagaggttggattggagtggttgagctataaaatttagaggcgcgccgtatcgagttttcacatggaagtcaaagcgtacagtgcgagcttgtacgttggtcttagtatcccacaagcttctgtctaggtatgatgatggctataagtcacccaaggcagaactcatcttgaagattgtctagagtgattttaccgctgatgaaatgactggactccctcctcctgctcttatacgaaaaattgcctgactctgcaaaggttgtttgtcttggaagatgatgtgcccccccatcgctcttatctcataccccgccatctttctagattctcatcttcaacaagaggggcaatccatgatctgcgatccagatgtgcttctggcctcatactctgccttcaggttgatgttcacttaattggtgacgaattcagctgatttgctgcagtatgctttgtgttggttctttccaggcttgtgccagccatgagcgctttgagagcatgttgtcacctataaactcgagtaacggccacatattgttcactacttgaatcacatacctaattttgatagaattgacatgtttaaagagctgaggtagctttaatgcctctgaagtattgtgacacagcttctcacagagtgagaatgaaaagttggactccccctaatgaagtaaaagtttcgtctctgaacggtgaagagcatagatccggcatcaactacctggctagactacgacgtcaattctgcggccttttgacctttatatatgtccattaatgcaatagattcttttttttttttttttttttttttttttttttttgcccaatttcgcagatcaaagtggacgttatagcatcataactaagctcagttgctgagggaagccgtctactaccttagcccatccatccagctccataccttgatactttagacgcgaagcaattcacactgtacgtctcgcagctctccttcccgctcttgcttccccactggggtccatggtgcgtgtatcgtcccctccttaat

SEQ ID NO: 15 - DNA, 615 bp, synthetic construct
  misc_feature 1..615: note = manual annotation of CRC17988 CDS
  source 1..615: mol_type = other DNA
atgaagttctccgcctccgttctcctgtccgccgccgcggcggtcctggcagcacctttccctgagccggctcctggtatccccagcgcgtccaccgccaggacgctgctcgccggcctccaggttgccgcttcggggagcgggaccggctacagtcgcgagaagttcccccactgggcgaccatcagcggcacttgcaacgcccgcgagtttgtgctgaagcgcgatggcgagaacgtcgtggtcaactcggcctgcaccgccgagtcgggcacctggaagagcccgtacgacggcgcctcctggactcaggccagcgacattgacattgaccacatggtccccctcaagaacgcctggatctctggagcctcctcgtggaccacggaccagcgcaagggcttcgccaacgatattacgcgcccgcagctctgggccgtgaccgacaatgtcaaccaggccaagagcgactcgagccccgacaggtggaagccgccgctgacgagcttctactgcacctatgctaagagctgggtgcaggtgaagagctactacaagctgaccgtcaccgatgccgaaaagggggccctgtcaggcatgctggacagctgctga

SEQ ID NO: 16 - protein, 205 aa, synthetic construct
  REGION 1..205: note = synthetic CRC17988 sequence
MKFSASVLLSAAAAVLAAPFPEPAPPGIPSASTARTLLAGLQVAASGSGTGYSREKFPHWATISGTCNAREFVLKRDGENVVVNSACTAESGTWKSPYDGASWTQASDIDIDHMVPLKNAWISGASSWTTDQRKGFANDITRPQLWAVTDNVNQAKSDSSPDRWKPPLTSFYCTYAKSWVQVKSYYKLTVTDAEKGALSGMLDSC

SEQ ID NO: 17 - protein, 188 aa, synthetic construct; identical to SEQ ID NO: 10
  REGION 1..188: note = synthetic CRC17988 mature sequence